(19) United States
(12) Patent Application Publication
(10) Pub. No.: US 2019/0183372 A1
(43) Pub. Date: Jun. 20, 2019

(54) Multiple Configuration Electrophysiological Mapping Catheter, and Systems, Devices, Components and Methods Associated Therewith

(71) Applicant: Ablacon Inc., Wheat Ridge, CO (US)

(72) Inventor: Peter Ruppersberg, Blonay (CH)

(21) Appl. No.: 16/156,637

(22) Filed: Oct. 10, 2018

Related U.S. Application Data
(63) Continuation-in-part of application No. 15/258,410, filed on Sep. 7, 2016, now Pat. No. 10,143,374.
Application No. 15/258,410 is a continuation-in-part of application No. 15/577,924, filed on Nov. 29, 2017.
Application No. 15/577,924 is a continuation-in-part of application No. 15/793,594, filed on Oct. 25, 2017.

(51) Int. Cl. A61B 5/042; A61B 18/14; A61B 5/00; A61B 34/20

(52) U.S. Cl. CPC A61B 5/0422; A61B 18/1492; A61B 2018/00839; A61B 34/20; A61B 5/6859; A61B 5/6858

(57) ABSTRACT

Disclosed are various examples and embodiments of a multiple configuration electrophysiological (EP) mapping catheter, and systems, devices, components and methods associated therewith. In some embodiments, the catheter is capable of being controllably deployed by a user inside or near a patient's heart in different geometric configurations according to the particular EP sensing and ablation requirements and needs at hand. For example, in some embodiments one and the same EP mapping catheter can be used to sense localized electrical signals originating in or near a patient's pulmonary vein or artery, and also to sense high-or-medium-spatial resolution electrical signals in the patient's atrium. In some embodiments, the electrode mapping assembly of one and the same EP mapping catheter is capable of assuming mushroom, fan- or paddle-shaped, and/or basket configurations, and thus eliminates the need to employ multiple different types of EP mapping catheters inside a patient's heart during, for example, an intravascular atrial fibrillation surgery and treatment session.

### RELATED APPLICATIONS

This application is a continuation-in-part of, and claims priority and other benefits from, the following U.S. patent applications: (a) U.S. patent application Ser. No. 15/258,410 filed on Sep. 7, 2016 entitled “Systems, Devices, Components and Methods for Detecting the Locations of Sources of Cardiac Rhythm Disorders in a Patient's Heart” to Ruppersberg (the ‘410 patent application”); (b) U.S. patent application Ser. No. 15/577,924 filed on Nov. 29, 2017 entitled “Optical Force Sensing Assembly for an Elongated Medical Device” to Ruppersberg (the ‘924 patent application”); and (c) U.S. patent application Ser. No. 15/793,594 filed on Oct. 25, 2017 entitled “Improved Electrophysiological Mapping Catheter” to Ruppersberg (the ‘594 patent application”). The respective entireties of the '410, '924, and '594 patent applications are hereby incorporated by reference herein.

### FIELD OF THE INVENTION

Various embodiments described and disclosed herein relate to the field of medicine generally, and more particularly to diagnosing and treating cardiac rhythm disorders in a patient's heart using electrophysiological (EP) mapping systems, EP mapping devices such as ablation catheters and EP mapping catheters, and EP mapping components and techniques, procedures and methods.

### BACKGROUND

Atrial fibrillation (or AF) is the most common type of heart arrhythmia or cardiac rhythm disorder. In atrial fibrillation, normal beating in the atria of the heart is irregular, and blood flow from the atria to the ventricles is compromised. Millions of people in the United States have AF. With the aging of the U.S. population, even more people will develop AF. Approximately 2% of people younger than age 65 have AF, while about 9% of people aged 65 years or older have AF. In some cases AF is treated with drugs. In other cases, external electrical shocks (electrical cardioversion) are delivered to the patient's heart. Open heart surgery can also be performed on a patient to treat AF.

Persistent atrial fibrillation (AF) is often caused by structural changes in atrial tissue, which can manifest themselves as multiwavelet re-entry and/or stable rotor mechanisms (see, e.g., De Groot M S et al., “Electropathological Substrate of Longstanding Persistent Atrial Fibrillation in Patients with Structural Heart Disease Epicardial Breakthrough,” Circulation, 2010, 3: 1674-1682). Radio frequency (RF) ablation targeting such host drivers of AF is generally accepted as one of the best therapeutic approaches to treating AF. RF ablation success rates in treating AF cases are currently limited, however, by a lack of sufficiently accurate and cost-effective diagnostic tools that are capable of quickly, cost-effectively, and precisely determining the source (or type), and location, of such AF drivers. Better diagnostic tools would help reduce the frequency and extent of cardiac ablation procedures to the minimum amount required to treat AF, and would help balance the benefits of decreased fibrillatory burden against the morbidity of increased lesion load.

What is needed are medical systems, devices, components and methods that can be employed to more quickly, efficiently, cost-effectively, and accurately diagnose and treat patients who have AF using intravascular techniques, where cardiac or pulmonary vein tissue is likely to be ablated, and where accurate and enhanced EP mapping of the heart can be carried out. What is also needed are improved means and methods of acquiring intracardiac electrogram signals that quickly, reliably and accurately yield the precise locations and sources of cardiac rhythm disorders in a patient's heart. Doing so would enable cardiac ablation procedures to be carried out with greater speed, greater locational precision, lower risk to the patient, reduced cost, and higher rates of success in treating cardiac rhythm disorders such as AF.

### SUMMARY

In one embodiment, there is provided a multiple configuration electrophysiological (EP) mapping catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first initial deployment configuration suitable for pulmonary vein isolation (PV) EP mapping; (b) a second intermediate deployment fan or paddle configuration suitable for high-resolution EP mapping; and (c) a third fully or nearly fully deployed basket configuration suitable for medium-resolution EP mapping, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and (iii) in the third configuration the electrode mapping assembly is deployed by the user a third distance from the distal portion of the catheter body, and further wherein the first distance is less than the second distance, the second distance is less than the third distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening.

In another embodiment, there is provided a method of deploying a multiple configuration EP mapping catheter in a patient, the catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first initial deployment configuration suitable for pulmonary vein isolation (PV) EP mapping; (b) a second intermediate deployment fan or paddle configuration suitable for high-resolution EP mapping; and (c) a third fully or nearly fully deployed basket configuration suitable for medium-resolution EP mapping, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and (iii) in the third configuration the electrode mapping assembly is deployed by the user a third distance from the distal portion of the catheter body, and further wherein the first distance is less than the second distance, the second distance is less than the third distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening, the method comprising two or more of: (1) deploying the electrode mapping assembly into the first configuration inside or near the patient's heart; (2) deploying the electrode mapping assembly into the second configuration inside or near the patient's heart, and (3) deploying the electrode mapping assembly into the third configuration inside or near the patient's heart.

In yet another embodiment, there is provided an EP mapping basket catheter comprising an elongated catheter body comprising a proximal portion, a distal portion, and a distal tip, an electrode deployment and control mechanism located near or at the proximal portion of the catheter body, a deployable electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into a basket configuration, the basket configuration having an imaginary central longitudinal axis associated therewith when the basket is deployed in an unobstructed and unconfined space, and further wherein an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening.

In still another embodiment, there is provided a method of deploying an EP mapping basket catheter in a patient, the catheter comprising an elongated catheter body comprising a proximal portion, a distal portion, and a distal tip, an electrode deployment and control mechanism located near or at the proximal portion of the catheter body, a deployable electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into a basket configuration, the basket configuration having an imaginary central longitudinal axis associated therewith when the basket is deployed in an unobstructed and unconfined space, and further wherein an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening, the method comprising deploying the electrode mapping assembly into the basket configuration inside or near the patient's heart.

In another embodiment, there is provided a multiple configuration EP mapping catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into the following configurations: (a) a first circular, semi-circular, oval, elliptical, or lasso-like configuration suitable for pulmonary vein isolation (PV) EP mapping; and (b) a second basket configuration, the basket having an imaginary central longitudinal axis associated therewith when the basket is deployed in an unobstructed and unconfined space, and further wherein: (i) in the first configuration the electrode mapping assembly is deployed by the user a first distance from the distal portion of the catheter body, and (ii) in the second configuration the electrode mapping assembly is deployed by the user a second distance from the distal portion of the catheter body; and further wherein the first distance is less than the second distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening.

In yet another embodiment, there is provided a method of deploying a multiple configuration EP mapping catheter in a patient, the catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into the following configurations: (a) a first circular, semi-circular, oval, elliptical, or lasso-like configuration suitable for pulmonary vein isolation (PV) EP mapping; and (b) a second basket configuration, the basket having an imaginary central longitudinal axis associated therewith when the basket is deployed in an unobstructed and unconfined space, and further wherein: (i) in the first configuration the electrode mapping assembly is deployed by the user a first distance from the distal portion of the catheter body, and (ii) in the second configuration the electrode mapping assembly is deployed by the user a second distance from the distal portion of the catheter body; and further wherein the first distance is less than the second distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening, the method comprising at least one of (1) deploying the electrode mapping assembly into the first configuration inside or near the patient's heart, and (2) deploying the electrode mapping assembly into the second configuration inside or near the patient's heart.

In still another embodiment, there is provided a multiple spatial resolution EP mapping catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first fan-shaped configuration of the mapping electrode assembly wherein electrodes mounted on or attached to central portions of adjoining spines are separated from one another by distances ranging between about 0.25 cm and about 2 cm such that the EP mapping electrode assembly is configured to provide high spatial resolution EP data; and (b) a second basket configuration of the mapping electrode assembly wherein electrodes mounted on or attached to central portions of adjoining spines are separated from one another by distances ranging between about 1 cm and about 4 cm such that the EP mapping electrode assembly is configured to provide medium spatial resolution EP data, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and further wherein the first distance is less than the second distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening.

In yet another embodiment, there is provided a method of deploying a multiple spatial resolution EP mapping catheter in a patient, the catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first fan-shaped configuration of the mapping electrode assembly wherein electrodes mounted on or attached to central portions of adjoining spines are separated from one another by distances ranging between about 0.25 cm and about 2 cm such that the EP mapping electrode assembly is configured to provide high spatial resolution EP data; and (b) a second basket configuration of the mapping electrode assembly wherein electrodes mounted on or attached to central portions of adjoining spines are separated from one another by distances ranging between about 1 cm and about 4 cm such that the EP mapping electrode assembly is configured to provide medium spatial resolution EP data, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and further wherein the first distance is less than the second distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening, the method comprising at least one of (1) deploying the electrode mapping assembly into the first configuration inside or near the patient's heart, and (2) deploying the electrode mapping assembly into the second configuration inside or near the patient's heart.

In still yet another embodiment, there is provided an EP mapping catheter comprising an elongated catheter body comprising a proximal portion, a distal portion, and a distal tip, an electrode deployment and control mechanism located near or at the proximal portion of the catheter body, a deployable electrode mapping assembly operably connected to the electrode deployment and control mechanism, the electrode mapping assembly comprising a plurality of electrodes and a plurality of splines, each spline having a proximal end and a distal end, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into at least one of the following configurations: (a) a first circular, semi-circular, oval, elliptical, or lasso-like configuration suitable for pulmonary vein isolation (PV) EP mapping; (b) a second fan-shaped configuration of the mapping electrode assembly suitable for acquiring high-resolution EP data; and (c) a third basket configuration suitable for acquiring medium-resolution EP data. In such an embodiment, an opening between splines may—or may not—be included or provided in the catheters described herein. Methods of deploying and using the catheter according to such embodiments are also contemplated, as are catheters capable of assuming only one of the aforementioned three configurations (e.g., circular, fan-shaped, and basket configurations).

In still further embodiments, any of the above- or below-described catheters and corresponding methods can be modified such that there is no opening located between adjoining splines where portions of the catheter body located proximally from the distal tip can be moved by a user away from the longitudinal axis of the basket through such an opening.

The foregoing embodiments may further comprise one or more of: the catheter being configured to permit portions of the catheter body located proximally from the distal tip to be moved by the user away from the longitudinal axis of the basket in the direction of and through the opening; the catheter being configured to permit portions of the catheter body located proximally from the distal tip to be moved by the user away from the longitudinal axis of the basket in the direction of and outside the opening; the distal tip of the catheter being configured to be steerable or bent by the user; an outer slidable sheath configured to permit deployment of the electrode mapping assembly from the distal tip of the catheter; an outer slidable sheath that is steerable; a steerable sheath comprising a steerable distal end; an electrode mapping assembly comprising between 4 splines and 12 splines; each spline having attached thereto, mounted thereon or formed therein between 1 and 16 electrodes; distal ends of adjoining splines forming pairs of splines that are joined or connected to one another; one or more navigation elements, navigation coils, navigation markers or navigation electrodes; a shape memory material comprising one or more of Nitinol, a shape memory metal, a shape memory alloy, a shape memory polymer, a shape memory composite, or a shape memory hybrid; at least one spline in the electrode mapping assembly comprising laminated materials; the mapping electrode assembly being deployed by pushing the mapping electrode assembly out of the distal end of the catheter using the electrode deployment and control mechanism; a tissue ablation mechanism located at or near the distal tip of the catheter; spatial resolution provided by the electrodes in the electrode mapping assembly and an associated spacing between splines changing in accordance with the first, second and third configurations thereof; a diameter of the arms of the electrode mapping assembly ranging between about 6 mm and about 14 mm when the electrode mapping assembly is deployed in the first configuration; a diameter of the arms of the electrode mapping assembly ranging between about 6 mm and about 14 mm when the electrode mapping assembly is deployed in the first configuration; a diameter of the arms of the electrode mapping assembly ranging between about 10 mm and about 20 mm when the electrode mapping assembly is deployed in the first configuration; a length of each tendon or chord ranging between about 6 mm and about 20 mm; the electrodes being one or more of unipolar electrodes and bipolar electrodes; spacing between adjoining electrodes located on the same spline ranging between about 0.5 mm and about 1 mm, between about 0.25 mm and about 2 mm, between about 6 mm and about 20 mm, between about 8 mm and about 18 mm, or between about 10 mm and about 15 mm; the third basket structure having an outer diameter ranging between about 20 mm and about 200 mm, between about 30 mm and about 100 mm in diameter, between about 40 mm and about 80 mm in diameter, or between about 50 mm and about 70 mm, or is about 50 mm, about 60 mm or about 70 mm.

The foregoing embodiments may further comprise one or more of: the distal tip of the catheter being configured to be steerable or bent by the user, and the user bends or steers the distal tip of the catheter inside or near the patient's heart; acquiring EP signals from the patient using electrodes in the deployed electrode mapping assembly; processing the acquired EP signals so that the signals may be interpreted by the user; redeploying the electrode mapping assembly into a different configuration or location within or near the patient's heart based upon results provided by the processed EP signals; changing the configuration of the electrode mapping assembly from one of the first, second and third configurations to a different configuration; deploying the mapping electrode assembly by pushing the mapping electrode assembly out of the distal end of the catheter using the electrode deployment and control mechanism; ablating tissue at a location in or near the patient's heart, the location being identified using the processed EP signals.

Further embodiments are disclosed herein or will become apparent to those skilled in the art after having read and understood the claims, specification and drawings hereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Different aspects of the various embodiments will become apparent from the following specification, drawings and claims in which:

FIG. 1(a) shows one embodiment and example of a combined cardiac electrophysiological mapping (EP), pacing and ablation system 100;

FIG. 1(b) shows one embodiment and example of computer system 300;

FIG. 2 illustrates some of the problems that can arise with conventional basket catheters, such as spline bunching and inadequate electrode coverage;

FIG. 3 shows an illustrative view of one embodiment of a distal portion of catheter 110 inside a patient's left atrium 14;

FIGS. 4(a) through 4(d) illustrate one embodiment of an EP mapping catheter 110;

FIGS. 5(a) through 5(d) illustrate another embodiment of an EP mapping catheter 110;

FIGS. 6(a) and 6(b) illustrate one embodiment of distal portion 108 of catheter 110 having mapping electrode assembly 120 initially deployed in a restricted or mushroom-shaped configuration, in two circular-shaped configurations and stages;

FIG. 7 illustrates one embodiment of distal portion 108 of catheter 110, where mapping electrode assembly 120 has been deployed in an intermediate fan- or paddle-shaped configuration extending further outwardly and backwardly from distal tip 112 with respect to the deployments of mapping electrode assemblies 120 shown in FIGS. 6(a) and 6(b).

FIG. 8 illustrates one embodiment of mapping electrode assembly 120 of FIGS. 6(a), 6(b) and 7 in a fully or nearly fully deployed basket configuration, where splines 126 have been pushed outwardly and backwardly fully from distal tip 112;

FIGS. 9 and 10 show front and side perspective views according to one embodiment of fully deployed mapping electrode assembly 120 of FIG. 8.

FIG. 11 shows one embodiment of distal portion 108 of catheter 110, where mapping electrode assembly 120 is in a fully deployed configuration, and where splines 126 have been pushed outwardly and backwardly fully from distal tip 112.

FIG. 12 illustrates one embodiment of mapping electrode assembly 120 fully deployed and electrically coupled to the walls of patient's left atrium 14;

FIG. 13 illustrates a conventional basket catheter mapping electrode assembly fully deployed inside a patient's atrium 14, and

FIG. 14 illustrates one method 200 of using the configurable multi-application electrophysiological mapping catheter 110.

The drawings are not necessarily to scale. Like numbers refer to like parts or steps throughout the drawings.

### DETAILED DESCRIPTIONS OF SOME EMBODIMENTS

Disclosed herein are various embodiments of systems, devices, components and methods for diagnosing and treating cardiac rhythm disorders in a patient's heart using EP mapping and ablation catheters, as well as EP imaging, navigation, and other types of medical systems, devices, components, and methods. Various embodiments described and disclosed herein also relate to systems, devices, components and methods for discovering with enhanced precision the location(s) of the source(s) of different types of cardiac rhythm disorders and irregularities. Such cardiac rhythm disorders and irregularities, include, but are not limited to, arrhythmias, atrial fibrillation (AF or A-fib), atrial tachycardia, atrial flutter, paroxysmal fibrillation, paroxysmal flutter, persistent fibrillation, ventricular fibrillation (V-fib), ventricular tachycardia, atrial tachycardia (A-tach), ventricular tachycardia (V-tach), supraventricular tachycardia (SVT), paroxysmal supraventricular tachycardia (PSVT), Wolff-Parkinson-White syndrome, bradycardia, sinus bradycardia, ectopic atrial bradycardia, junctional bradycardia, heart blocks, atrioventricular block, idioventricular rhythm, areas of fibrosis, breakthrough points, focus points, re-entry points, premature atrial contractions (PACs), premature ventricular contractions (PVCs), and other types of cardiac rhythm disorders and irregularities.

Also described herein is an EP mapping catheter that is capable of assuming multiple configurations within or near a patient's heart. These multiple configurations permit a single catheter to electrographically image a patient's atrium and portions of the PV near the atrium at different resolutions, all using the same EP mapping catheter. Following initial EP mapping of a patient's atrium and/or PV with the EP mapping catheter, the same EP mapping catheter can be then used to detect PV isolation and extra PV sources following ablation, and can also be used to provide high resolution recordings from major portions of the atrium. In some embodiments, the EP mapping catheter includes or operates in conjunction with an ablation catheter.

In the following description, for purposes of explanation, numerous specific details are set forth in order to provide a thorough understanding of example embodiments or aspects. It will be evident, however, to those skilled in the art that an example embodiment may be practiced without necessarily using all of the disclosed specific details, and that other embodiments not specifically or wholly disclosed are also contemplated and fall within the scope of the various inventions.

Before discussing in detail some of the various embodiments of the unique configurable multi-application electrophysiological mapping catheter disclosed and described herein, several aspects of systems, devices, components and methods that may be employed in conjunction with catheters are first described and disclosed.

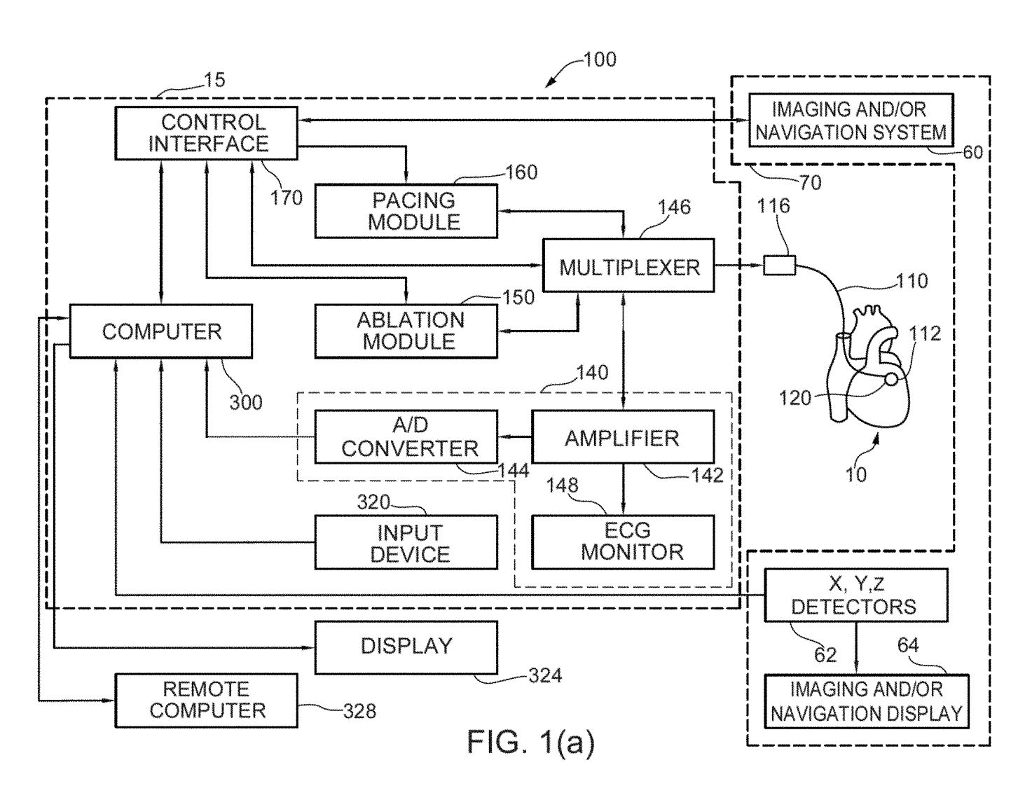

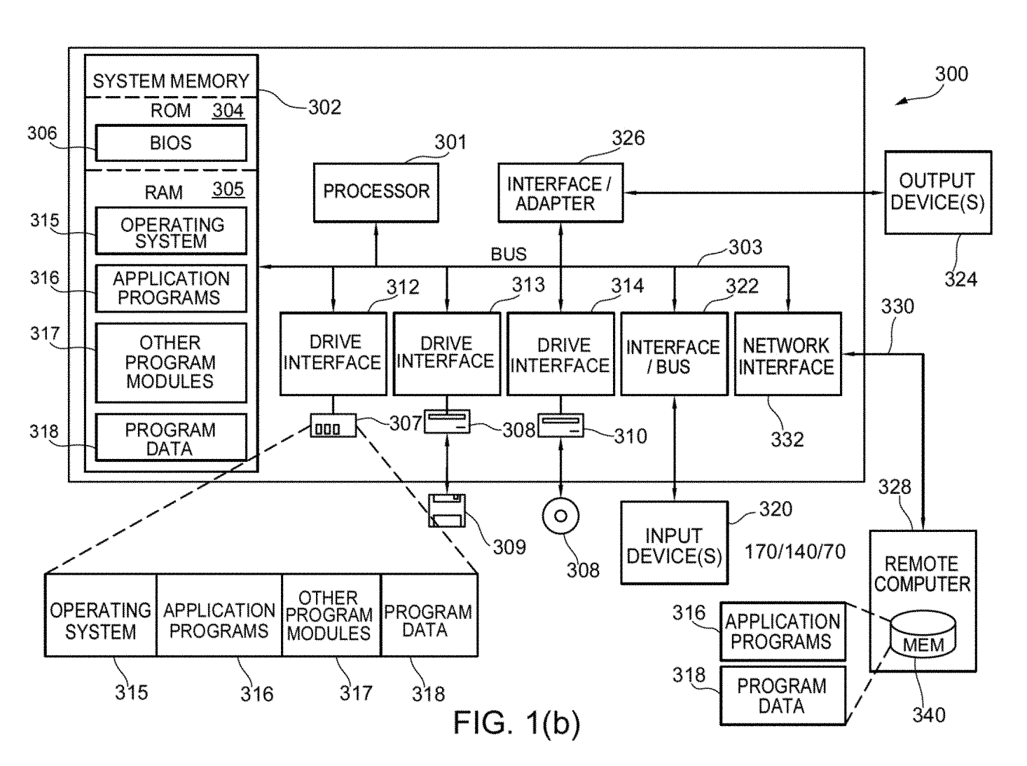

Referring now to FIG. 1(a), there is illustrated one embodiment of a combined cardiac electrophysiological mapping (EP), pacing and ablation system 100. Note that in some embodiments system 100 may not include ablation module 150 and/or pacing module 160. Among other things, the embodiment of system 100 shown in FIG. 1(a) is configured to detect and reconstruct cardiac activation information acquired from a patient's heart relating to cardiac rhythm disorders and/or irregularities, and is further configured to detect and discover the location of the source of such cardiac rhythm disorders and/or irregularities with enhanced precision relative to prior art techniques and devices. In some embodiments, system 100 is further configured to treat the location of the source of the cardiac rhythm disorder or irregularity, for example by ablating the patient's heart at the detected location.

The embodiment of system 100 shown in FIG. 1(a) comprises five main functional units: electrophysiological mapping (EP mapping unit) 140 (which is also referred to herein as data acquisition device 140), ablation module 150, pacing module 160, imaging and/or navigation system 70, and computer or computing device 300. A data acquisition, processing and control system can be configured to comprise data acquisition device 140, ablation module 150, pacing module 160, control interface 170 and computer or computing device 300. In one embodiment, at least one computer or computing device or system 300 is employed to control the operation of one or more of systems, modules and devices 140, 150, 160, 170 and 70. Alternatively, the respective operations of systems, modules or devices 140, 150, 160, 170 and 70 may be controlled separately by each of such systems, modules and devices, or by some combination of such systems, modules and devices.

Computer or computing device 300 may be configured to receive operator inputs from an input device 320 such as a keyboard, mouse and/or control panel. Outputs from computer 300 may be displayed on display or monitor 324 or other output devices (not shown in FIG. 1(a)). Computer 300 may also be operably connected to a remote computer or analytic database or server 328. At least each of components, devices, modules and systems 60, 110, 140, 146, 148, 150, 170, 300, 324 and 328 may be operably connected to other components or devices by wireless (e.g., Bluetooth) or wired means. Data may be transferred between components, devices, modules or systems through hardwiring, by wireless means, or by using portable memory devices such as USB memory sticks.

During electrophysiological (EP) mapping procedures, multi-electrode catheter 110 is typically introduced percutaneously into the patient's heart 10. Catheter 110 is passed through a blood vessel (not shown), such as a femoral vein or the aorta, and thence into an endocardial site such as the atrium or ventricle of the heart 10, or nearby pulmonary vein(s).

It is contemplated that other catheters, including other types of mapping or EP catheters, lasso catheters, pulmonary vein isolation (PVI) ablation catheters (which can operate in conjunction with lasso and other types of sensing catheters), ablation catheters, navigation catheters, and still other types of EP mapping catheters such as EP monitoring catheters and spiral catheters, may also be introduced into the heart, and that additional surface electrodes may be attached to the skin of the patient to record electrocardiograms (ECGs).

When system 100 is operating in an EP mapping mode, multi-electrode catheter 110 functions as a detector of intra-electrocardiac signals, while optional surface electrodes may serve as detectors of surface ECGs. In one embodiment, the analog signals obtained from the intracardiac and/or surface electrodes are routed by multiplexer 146 to data acquisition device 140, which comprises an amplifier 142 and an A/D converter (ADC) 144. The amplified or conditioned electrogram signals may be displayed by electrocardiogram (ECG) monitor 148. The analog signals are also digitized via ADC 144 and input into computer 300 for data processing, analysis and graphical display.

In one embodiment, catheter 110 is configured to detect cardiac activation information in the patient's heart 10, and to transmit the detected cardiac activation information to data acquisition device 140, either via a wireless or wired connection. In one embodiment that is not intended to be limiting with respect to the number, arrangement, configuration, or types of electrodes, catheter 110 includes a plurality of 64 electrodes, probes and/or sensors A1 through H8 arranged in an 8×8 grid that are included in electrode mapping assembly 120, which is configured for insertion into the patient's heart through the patient's blood vessels and/or veins. Other numbers, arrangements, configurations and types of electrodes in catheter 110 are, however, also contemplated, such as by way of non-limiting example, 8, 16, 24, 32, 48, 96 and/or 124 electrodes being included in electrode mapping assembly 120. In many embodiments, at least some electrodes, probes and/or sensors included in catheter 110 are configured to detect cardiac activation or electrical signals, and to generate electrocardiograms or electrogram signals, which are then relayed by electrical conductors from or near the distal end of catheter 110 to proximal portion 116 of catheter 110 to data acquisition device 140.

Note that in many embodiments of system 100, multiplexer 146 acting as an arbiter between sub-systems or modules 60, 140, 150, 160, and 300 is not employed for various reasons. In some embodiments of system 100, separate sub-systems are provided for each of EP data acquisition device 140, ablation module 150, pacing module 160, imaging and/or navigation system 60, computer system 300, and so on. The embodiment shown in FIG. 1(a) is can thus be viewed as an illustrative overview of how the various sub-systems may function and work together. Thus, and by way of non-limiting example, in some embodiments, multiplexer 146 is separate from catheter 110 and data acquisition device 140. In other embodiments, multiplexer 146 is combined in catheter 110 or data acquisition device 140. In still other embodiments, multiplexer 146 is not employed at all.

In one embodiment, a medical practitioner or health care professional employs catheter 110 as a roving catheter to locate the site of the location of the source of a cardiac rhythm disorder or irregularity in the endocardium quickly and accurately, without the need for open-chest and open-heart surgery. In one embodiment, this is accomplished by using multi-electrode catheter 110 in combination with real-time or near-real-time data processing and interactive display by computer 300, and optionally in combination with imaging and/or navigation system 70. In one embodiment, multi-electrode catheter 110 deploys at least a two-dimensional array of electrodes against a site of the endocardium at a location that is to be mapped, more about which is said below. The intracardiac or electrogram signals detected by the catheter's electrodes provide data sampling of the electrical activity in the local site spanned by the array of electrodes.

In one embodiment, the electrogram signal data are processed by computer 300 to produce a display showing the locations(s) of the source(s) of cardiac rhythm disorders and/or irregularities in the patient's heart 10 in real-time or near-real-time, further details of which are provided below. That is, at and between the sampled locations of the patient's endocardium, computer 300 may be configured to compute and display in real-time or near-real-time an estimated, detected and/or determined location(s) of the site(s), source(s) or origin)s) of the cardiac rhythm disorder(s) and/or irregularity(s) within the patient's heart 10. This permits a medical practitioner to move interactively and quickly the electrodes of catheter 110 towards the location of the source of the cardiac rhythm disorder or irregularity.

In some embodiments of system 100, one or more electrodes, sensors or probes detect cardiac activation from the surface of the patient's body as surface ECGs, or remotely without contacting the patient's body (e.g., using magnetocardiograms). In another example, some electrodes, sensors or probes may derive cardiac activation information from echocardiograms. In various embodiments of system 100, external or surface electrodes, sensors and/or probes can be used separately or in different combinations, and further may also be used in combination with intracardiac electrodes, sensors and/or probes inserted within the patient's heart 10. Many different permutations and combinations of the various components of system 100 are contemplated having, for example, reduced, additional or different numbers of electrical sensing and other types of electrodes, sensors and/or transducers.

Continuing to refer to FIG. 1(a), in one embodiment EP mapping system or data acquisition device 140 is configured to condition the analog electrogram signals delivered by catheter 110 from electrodes A1 through H8 in amplifier 142. Conditioning of the analog electrogram signals received by amplifier 142 may include, but is not limited to, low-pass filtering, high-pass filtering, bandpass filtering, and notch filtering. The conditioned analog signals are then digitized in analog-to-digital converter (ADC) 144. ADC 144 may further include a digital signal processor (DSP) or other type of processor which is configure to further process the digitized electrogram signals (e.g., low-pass filter, high-pass filter, bandpass filter, notch filter, automatic gain control, amplitude adjustment or normalization, artifact removal, etc.) before they are transferred to computer or computing device 300 for further processing and analysis.

In some embodiments, the rate at which individual electrogram and/or ECG signals are sampled and acquired by system 100 can range between about 0.25 milliseconds and about 8 milliseconds, and may be about 0.5 milliseconds, about 1 millisecond, about 2 milliseconds or about 4 milliseconds. Other sample rates are also contemplated. While in some embodiments system 100 is configured to provide unipolar signals, in other embodiments system 100 is configured to provide bipolar signals.

In one embodiment, system 100 can include a BARD® LABSYSTEM™ PRO EP Recording System, which is a computer and software driven data acquisition and analysis tool designed to facilitate the gathering, display, analysis, pacing, mapping, and storage of intracardiac EP data. Also in one embodiment, data acquisition device 140 can include a BARD® CLEARSIGN™ amplifier, which is configured to amplify and condition electrocardiographic signals of biologic origin and pressure transducer input, and transmit such information to a host computer (e.g., computer 300 or another computer).

As shown in FIG. 1(a), and as described above, in some embodiments system 100 includes ablation module 150, which may be configured to deliver RF ablation energy through catheter 110 and corresponding ablation electrodes disposed near distal end 112 thereof, and/or to deliver RF ablation energy through a different catheter (not shown in FIG. 1(a)). Suitable ablation systems and devices include, but are not limited to, cryogenic ablation devices and/or systems, radiofrequency ablation devices and/or systems, ultrasound ablation devices and/or systems, high-intensity focused ultrasound (HIFU) devices and/or systems, chemical ablation devices and/or systems, and laser ablation devices and/or systems.

When system 100 is operating in an ablation mode, multi-electrode catheter 110 fitted with ablation electrodes, or a separate ablation catheter, is energized by ablation module 150 under the control of computer 300, control interface 170, and/or another control device or module. For example, an operator may issue a command to ablation module 150 through input device 320 to computer 300. In one embodiment, computer 300 or another device controls ablation module 150 through control interface 170. Control of ablation module 150 can initiate the delivery of a programmed series of electrical energy pulses to the endocardium via catheter 110 (or a separate ablation catheter, not shown in FIG. 1(a)). One embodiment of an ablation method and device is disclosed in U.S. Pat. No. 5,383,917 to Desai et al., the entirety of which is hereby incorporated by reference herein.

In an alternative embodiment, ablation module 150 is not controlled by computer 300, and is operated manually directly under operator control. Similarly, pacing module 160 may also be operated manually directly under operator control. The connections of the various components of system 100 to catheter 110, to auxiliary catheters, or to surface electrodes may also be switched manually or using multiplexer 146 or another device or module.

When system 100 is operating in an optional pacing mode, multi-electrode catheter 110 is energized by pacing module 160 operating under the control of computer 300 or another control device or module. For example, an operator may issue a command through input device 320 such that computer 300 controls pacing module 160 through control interface 170, and multiplexer 146 initiates the delivery of a programmed series of electrical simulating pulses to the endocardium via the catheter 110 or another auxiliary catheter (not shown in FIG. 1(a)). One embodiment of a pacing module is disclosed in M. E. Josephson et al., in “VENTRICULAR ENDOCARDIAL PACING II, The Role of Pace Mapping to Localize Origin of Ventricular Tachycardia,” The American Journal of Cardiology, vol. 50, November 1982.

Computing device or computer 300 is appropriately configured and programmed to receive or access the electrogram signals provided by data acquisition device 140. Computer 300 is further configured to analyze or process such electrogram signals in accordance with the methods, functions and logic disclosed and described herein so as to permit reconstruction of cardiac activation information from the electrogram signals. This, in turn, makes it possible to locate with at least some reasonable degree of precision the location of the source of a heart rhythm disorder or irregularity. Once such a location has been discovered, the source may be eliminated or treated by means that include, but are not limited to, cardiac ablation.

In one embodiment, and as shown in FIG. 1(a), system 100 also comprises a physical imaging and/or navigation system 70. Physical imaging and/or navigation device 60 included in system 70 may be, by way of example, a 2- or 3-axis fluoroscope system, an ultrasonic system, a magnetic resonance imaging (MRI) system, a computed tomography (CT) imaging system, and/or an electrical impedance tomography EIT) system. Operation of system 70 be controlled by computer 300 via control interface 170, or by other control means incorporated into or operably connected to imaging or navigation system 70. In one embodiment, computer 300 or another computer triggers physical imaging or navigation system 60 to take “snap-shot” pictures of the heart 10 of a patient (body not shown). A picture image is detected by a detector 62 along each axis of imaging, and can include a silhouette of the heart as well as a display of the inserted catheter 110 and its sensing electrodes, which is displayed on imaging or navigation display 64. Digitized image or navigation data may be provided to computer 300 for processing and integration into computer graphics that are subsequently displayed on monitor or display 64 and/or 324.

In one embodiment, system 100 further comprises or operates in conjunction with catheter or electrode position transmitting and/or receiving coils or antennas located at or near the distal end of an EP mapping catheter 110, or that of an ablation or navigation catheter 110, which are configured to transmit electromagnetic signals for intra-body navigational and positional purposes.

In one embodiment, imaging or navigation system 70 is used to help identify and determine the precise two- or three-dimensional positions of the various electrodes included in catheter 110 within patient's heart 10, and is configured to provide electrode position data to computer 300. Electrodes, position markers, and/or radio-opaque markers can be located on various portions of catheter 110, mapping electrode assembly 120 and/or distal end 112, or can be configured to act as fiducial markers for imaging or navigation system 70.

Medical navigation systems suitable for use in the various embodiments described and disclosed herein include, but are not limited to, image-based navigation systems, model-based navigation systems, optical navigation systems, electromagnetic navigation systems (e.g., BIOSENSE® WEBSTER® CARTO® system), and impedance-based navigation systems (e.g., the St. Jude® ENSITE™ VELOCITY™ cardiac mapping system), and systems that combine attributes from different types of imaging AND navigation systems and devices to provide navigation within the human body (e.g., the MEDTRONIC® STEALTHSTATION® system).

In view of the structural and functional descriptions provided herein, those skilled in the art will appreciate that portions of the described devices and methods may be configured as methods, data processing systems, or computer algorithms. Accordingly, these portions of the devices and methods described herein may take the form of a hardware embodiment, a software embodiment, or an embodiment combining software and hardware, such as shown and described with respect to computer system 300 illustrated in FIG. 1(b). Furthermore, portions of the devices and methods described herein may be a computer algorithm or method stored in a computer-usable storage medium having computer readable program code on the medium. Any suitable computer-readable medium may be utilized including, but not limited to, static and dynamic storage devices, hard disks, optical storage devices, and magnetic storage devices.

Certain embodiments of portions of the devices and methods described herein are also described with reference to block diagrams of methods, systems, and computer algorithm products. It will be understood that such block diagrams, and combinations of blocks diagrams in the Figures, can be implemented using computer-executable instructions. These computer-executable instructions may be provided to one or more processors of a general purpose computer, a special purpose computer, or any other suitable programmable data processing apparatus (or a combination of devices and circuits) to produce a machine, such that the instructions, which executed via the processor(s), implement the functions specified in the block or blocks of the block diagrams.

These computer-executable instructions may also be stored in a computer-readable memory that can direct computer 300 or other programmable data processing apparatus to function in a particular manner, such that the instructions stored in the computer-readable memory result in an article of manufacture including instructions which implement the function specified in an individual block, plurality of blocks, or block diagram. The computer program instructions may also be loaded onto computer 300 or other programmable data processing apparatus to cause a series of operational steps to be performed on the computer or other programmable apparatus to produce a computer implemented process such that the instructions which execute on computer 300 or other programmable apparatus provide steps for implementing the functions specified in the an individual block, plurality of blocks, or block diagram.

In this regard, FIG. 1(b) illustrates only one example of a computer system 300 (which, by way of example, can include multiple computers or computer workstations) that can be employed to execute one or more embodiments of the devices and methods described and disclosed herein, such as devices and methods configured to acquire and process sensor or electrode data, to process image data, and/or transform sensor or electrode data and image data associated with the analysis of cardiac electrical activity and the carrying out of the combined electrophysiological mapping and analysis of the patient's heart 10 and ablation therapy delivered thereto.

Computer system 300 can be implemented on one or more general purpose computer systems or networked computer systems, embedded computer systems, routers, switches, server devices, client devices, various intermediate devices/nodes or standalone computer systems. Additionally, computer system 300 or portions thereof may be implemented on various mobile devices such as, for example, a personal digital assistant (PDA), a laptop computer and the like, provided the mobile device includes sufficient processing capabilities to perform the required functionality.

In one embodiment, computer system 300 includes processing unit 301 (which may comprise a CPU, controller, microcontroller, processor, microprocessor or any other suitable processing device), system memory 302, and system bus 303 that operably connects various system components, including the system memory, to processing unit 301. Multiple processors and other multi-processor architectures also can be used to form processing unit 301. System bus 303 can comprise any of several types of suitable bus architectures, including a memory bus or memory controller, a peripheral bus, or a local bus. System memory 302 can include read only memory (ROM) 304 and random access memory (RAM) 305. A basic input/output system (BIOS) 306 can be stored in ROM 304 and contain basic routines configured to transfer information and/or data among the various elements within computer system 300.

Computer system 300 can include a hard disk drive 303, a magnetic disk drive 308 (e.g., to read from or write to removable disk 309), or an optical disk drive 310 (e.g., for reading CD-ROM disk 311 or to read from or write to other optical media). Hard disk drive 303, magnetic disk drive 308, and optical disk drive 310 are connected to system bus 303 by a hard disk drive interface 312, a magnetic disk drive interface 313, and an optical drive interface 314, respectively. The drives and their associated computer-readable media are configured to provide nonvolatile storage of data, data structures, and computer-executable instructions for computer system 300. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of the devices and methods described and disclosed herein.

A number of program modules may be stored in drives and RAM 303, including operating system 315, one or more application programs 316, other program modules 313, and program data 318. The application programs and program data can include functions and methods programmed to acquire, process and display electrical data from one or more sensors, such as shown and described herein. The application programs and program data can include functions and methods programmed and configured to process data acquired from a patient for assessing heart function and/or for determining parameters for delivering a therapy and/or assessing heart function, such as shown and described herein with respect to FIGS. 1-10(f).

A health care provider or other user may enter commands and information into computer system 300 through one or more input devices 320, such as a pointing device (e.g., a mouse, a touch screen, etc.), a keyboard, a microphone, a joystick, a game pad, a scanner, and the like. For example, the user can employ input device 320 to edit or modify the data being input into a data processing algorithm or method (e.g., only data corresponding to certain time intervals). These and other input devices 320 may be connected to processing unit 301 through a corresponding input device interface or port 322 that is operably coupled to the system bus, but may be connected by other interfaces or ports, such as a parallel port, a serial port, or a universal serial bus (USB). One or more output devices 324 (e.g., display, a monitor, a printer, a projector, or other type of display device) may also be operably connected to system bus 303 via interface 326, such as through a video adapter.

Computer system 300 may operate in a networked environment employing logical connections to one or more remote computers, such as remote computer 328. Remote computer 328 may be a workstation, a computer system, a router, or a network node, and may include connections to many or all the elements described relative to computer system 300. The logical connections, schematically indicated at 330, can include a local area network (LAN) and/or a wide area network (WAN).

When used in a LAN networking environment, computer system 300 can be connected to a local network through a network interface or adapter 332. When used in a WAN networking environment, computer system 300 may include a modem, or may be connected to a communications server on the LAN. The modem, which may be internal or external, can be connected to system bus 303 via an appropriate port interface. In a networked environment, application programs 316 or program data 318 depicted relative to computer system 300, or portions thereof, may be stored in a remote memory storage device 340.

Further information and details regarding acquisition, processing and interpretation of EP mapping data are disclosed and described in the co-pending '410 patent application. At least portions of the disclosure of the '410 patent application find application in catheter 110 disclosed and described herein.

Turning now to considerations relating specifically to the various embodiments of the unique, configurable, multiple application, electrophysiological mapping catheter described and disclosed herein, conventional surgical techniques and catheters for diagnosing and treating AF in a patient often involve using a series of different intravascular EP mapping catheters, in addition to an intravascular ablation catheter. These different intravascular EP mapping catheters are often employed in multiple successive procedures performed one after the other during a single surgical session.

In such a typical surgical session, a series of different types of EP mapping catheters and an ablation catheter are used first to sense or map the electrical signals in a patient's atrium with sensing electrodes, next to ablate or otherwise treat tissue in the atrium or at or near the pulmonary vein at locations where electrical anomalies have been detected, and then to confirm that ablation has disrupted or destroyed the electrical sources of the AF. During the surgical session, additional intravascular EP mapping and ablation procedures may be required so that the precise location(s) of sources of still-remaining errant AF signals may be determined, such remaining sources may be ablated, and the locations of sources of errant AF signals may be confirmed to have been ablated successfully.

One way to treat some forms of AF is carry out RF or cryogenic ablation that results in pulmonary vein isolation (PVI), which is carried out by performing multiple ablations in a circular path or pattern around the pulmonary veins (PVs) of the patient. Usually both PVs are ablated, with access being provided by the right and left atria, which are adjacent to the PVs. To validate that ablation has produced sufficient PVI in a patient, in many instances a LASSO® mapping catheter specifically designed to sense signals in or near the PVs, or an EP mapping basket catheter, such as a Boston Scientific CONSTELLATION catheter or a TOPERA FIRMap® catheter, is introduced into or near the PVs after the ablation procedure has been completed so as to detect any remaining channels of excitation indicated by atrial signals still arriving in the PVs (or arriving in the atrium from the PVs). Sometimes these catheters provide insufficient spatial resolution to map the precise locations of the sources of errant AF signals.

If still greater spatial resolution of errant AF signals is to be obtained, a different type of EP mapping catheter (e.g., a star-shaped, fan- or grid-type catheter) with decreased inter-electrode spacing can be guided to a target inserted site after the LASSO or basket catheter has been withdrawn from the patient. Once PVI has been confirmed to have been accomplished successfully, ablation in the right or left atria may be required if errant arrhythmias are still detected. EP mapping basket catheters are often employed to map the patient's atria following PVI. However, conventional basket catheters often do not optimally fit into or conform to the walls of the irregularly-shaped atria, and also are frequently incapable of providing high resolution recordings owing to irregular spacing of the splines containing the sensing electrode once the basket assembly has been positioned within the patient's heart. Additionally, medium resolution basket catheters are often difficult or impossible to position in a desired orientation and placement within a patient's atrium owing, among other things, to the steep angle of entrance into the atrium by the tip of the basket catheter through a trans-septal puncture, the long paths to the right and left atria (and consequent difficulty in adjusting or tweaking the position of the tip of a catheter and its electrodes therein), and the oblong and generally irregular geometries of the left and right atria As a result, EP mapping results obtained using conventional basket catheters can be suboptimal owing to insufficient and/or uneven electrode coverage, and to electrodes not being positioned in the locations required to acquire useful signals. In such instances, a star-shaped PENTARAY® or other high-resolution EP mapping catheters can be used to obtain higher resolution EP recordings. This, of course, requires the use and deployment of yet another type of EP mapping catheter, which as a result is often not done.

In the above-described intravascular surgical techniques, the following different types of intravascular catheters may thus be employed: (a) a lasso-type or circular electrode electrophysiological (EP) mapping catheter configured to sense electrical activity in and around the pulmonary veins; (b) a basket EP mapping catheter configured to sense electrical activity in and around the atrium at medium resolution; (c) a star-shaped, fan- or grid-type EP mapping catheter configured to sense electrical activity in and around the atrium at higher resolution; and (d) an RF, cryogenic or other type of ablation catheter configured to ablate tissue in the atria or pulmonary veins at locations that have been identified as the source(s) of the AF. Oftentimes an ablation catheter is deployed in the patient's heart at the same time that an EP mapping catheter is deployed therein.

According to the above-described surgical techniques, multiple different EP mapping catheters are thus inserted into and then withdrawn from the patient's heart, typically via the femoral vein. Up to four or more different catheters may be employed one after the other in a single surgical session to treat a patient's AF. Each catheter used in the session has a purchase price associated with it; most EP mapping catheters are used once only, and are thrown away after the session has ended. In addition, the greater the number of intravascular procedures performed on a patient, the greater the risk to the patient.

Problems that can and do occur using conventional basket catheters, such as spline bunching and poor electrode coverage, are described in considerable detail in the following publications: (a) “Basket-Type Catheters: Diagnostic Pitfalls Caused by Deformation and Limited Coverage” to Oesterlein et al., BioMed Research International, Volume 2016, Article ID 5340574 (“the Oesterlein publication”); (b) “Practical Considerations of Mapping Persistent Atrial Fibrillation With Whole-Chamber Basket Catheters” to Laughner et al., JACC: Clinical Electrophysiology, Volume 2, Issue 1, February 2016, Pages 55-65 (“the first Laughner publication”); and (c) “Atrial Mapping With Basket Catheters—A Basket Case?” to Hummel et al., JACC: Clinical Electrophysiology, Volume 2, Issue 1, February 2016, Pages 66-68 (“the Hummel publication”). The respective entireties of the Oesterlein, Laughner and Hummel publications, complete copies of which were submitted on the filing date corresponding to the present patent application, are incorporated by reference herein.

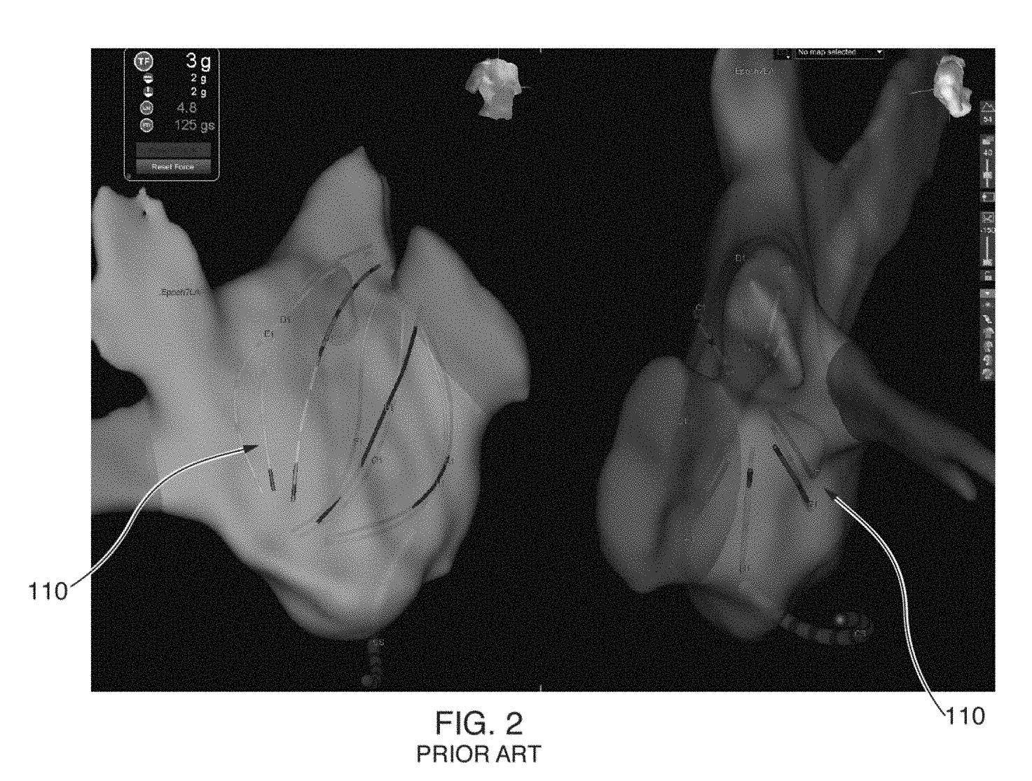

FIG. 2 illustrates some of the problems that can arise with conventional prior art basket catheters, and more particularly problems that can arise from spline bunching and inadequate electrode coverage. Shown in FIG. 2 are anterior (left side of FIG. 2) and posterior (right side of FIG. 2) views of a patient's left atrium with a prior art TOPERA FIRMap basket catheter deployed therein. The two images shown in FIG. 2 were acquired using a TOPERA RhythmView 3D mapping workstation. In FIG. 2, the eight splines of the FIRMap basket catheter are labelled A through H, and the eight electrodes on each spline are numbered 1 through 8 according to conventional nomenclature and practice. As shown, splines E and F are widely spaced from one another, while the other splines are more closely (or too closely) spaced from one another. A significant gap in EP mapping coverage resulted between splines E and F, as well as uneven electrode coverage in the remainder of the patient's left atrium. Once the catheter shown in FIG. 2 was emplaced within the patient's left atrium, moving it into a different position to obtain more even or better electrode coverage was difficult or impossible, as movement of the basket was effectively limited to minor adjustments of the basket forwards and backwards, or spinning or rotation of the basket within the patient's left atrium.

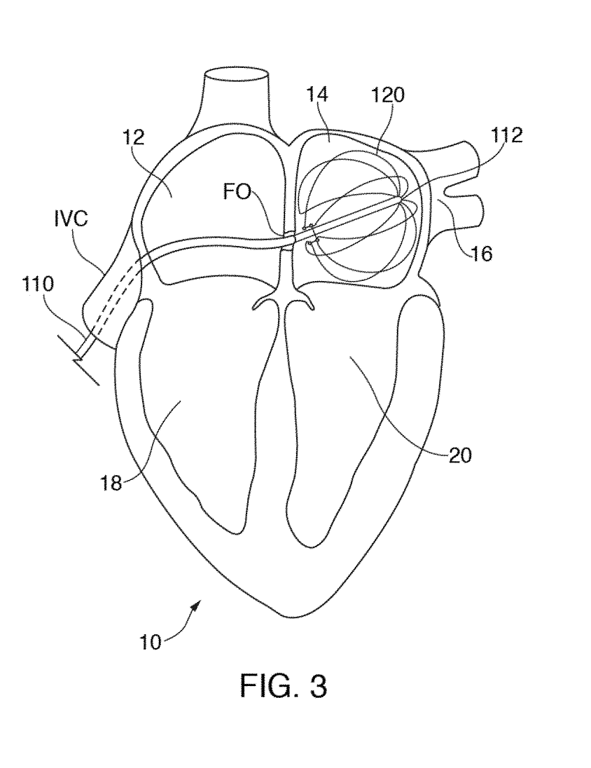

Referring now to FIG. 3, there is shown an illustrative view of one embodiment of a distal portion of catheter 110 inside a patient's left atrium 14. As shown in FIG. 2, heart 10 includes right atrium 12, left atrium 14, right ventricle 18, and left ventricle 20. Mapping electrode assembly 120 is shown in a fully deployed, expanded or open state inside left atrium 14 after it has been inserted through the patient's inferior vena cava and foramen ovalen (“IVC” and “FO” in FIG. 2), and in one embodiment is configured to obtain electrogram signals from left atrium 12 via electrodes 122 included in mapping electrode assembly 120. Mapping electrode assembly and catheter 110 may also be positioned within the patient's right atrium 12, left ventricle 18, and/or right ventricle 20. In FIG. 2, distal tip 112 of catheter 110 is punched through the FO and/or the trans-septal wall into the left atrium from the right atrium. The location and steep angle of approach provided by the resulting trans-septal puncture typically significantly restrict the freedom of movement and positionability that is possible using a conventional basket catheter after it has been deployed inside the left atrium. Contrariwise, owing to the unique structural attributes and configuration of the multi-configuration and application EP mapping catheter 110 described and disclosed herein, such as splines 126 bending backwardly from tip 112 in the proximal direction, positionability and maneuvering of distal portion 108 of catheter 112 are much enhanced.

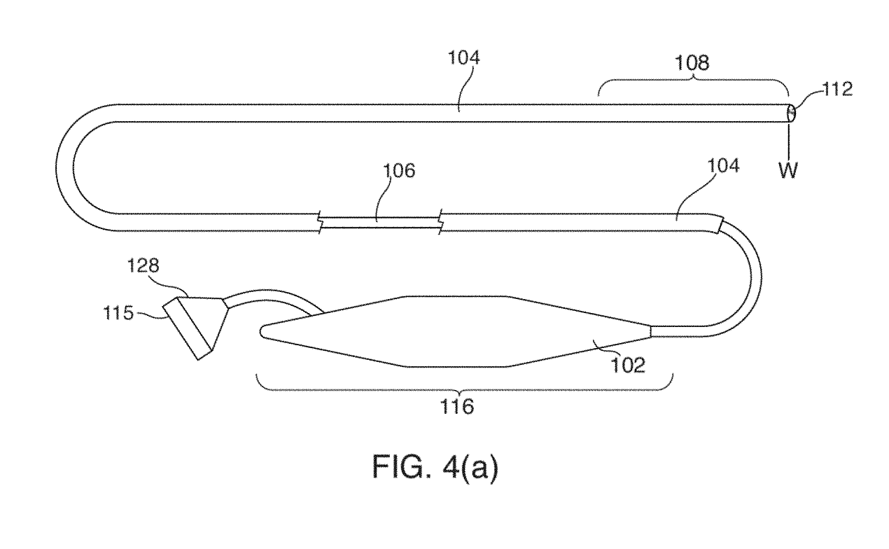

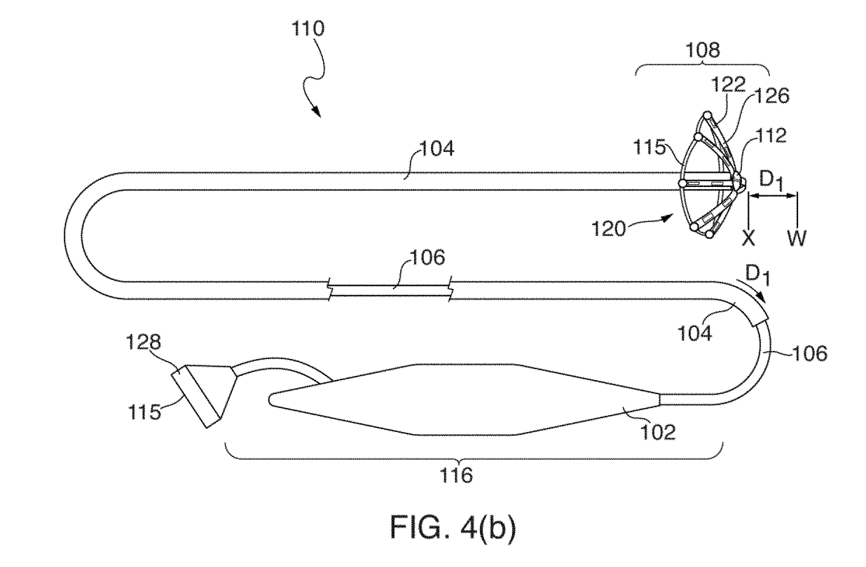

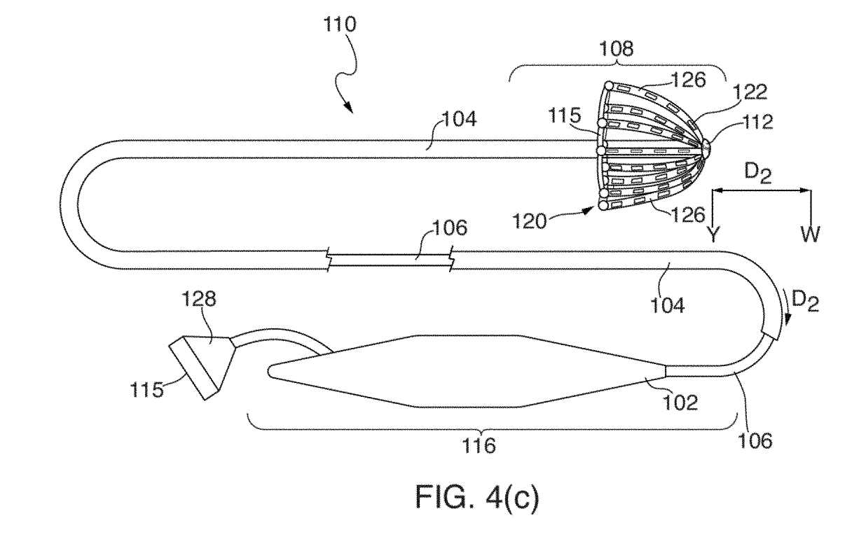

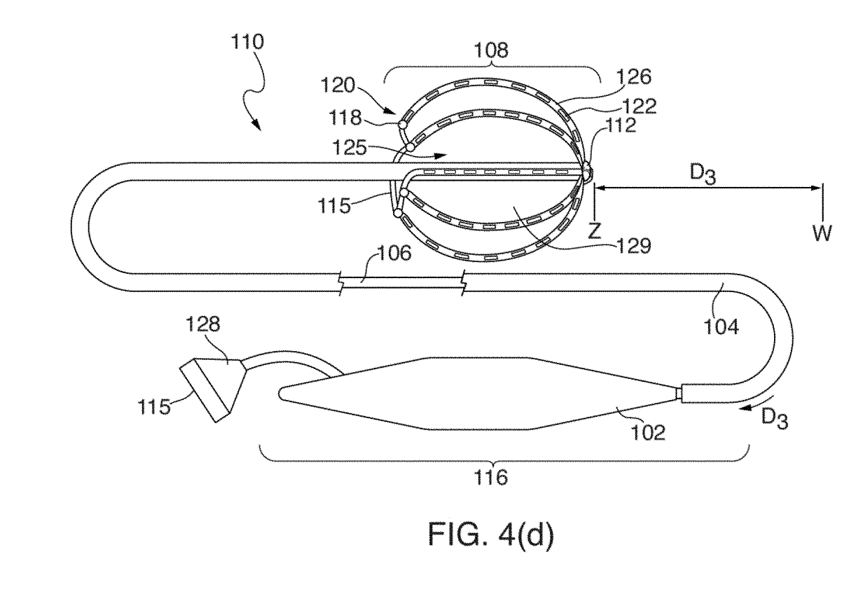

FIGS. 4(a) through 4(d) illustrate one embodiment of EP mapping catheter 110 comprising mapping electrode assembly 120 located at the distal portion 108 of catheter 110, where portions of catheter body 106 are covered by outer slidable sheath 104. In FIGS. 4(a) through 4(d), once distal end 112 of EP mapping catheter 110 has been guided to a desired location within patient's heart 10, mapping electrode assembly 120 can be deployed in one or more configurations within patient's heart 10 according to the physician's objective at hand (e.g., obtain EP recordings at or near the PV or inside atrium 12 or 14, and/or obtain low, medium or high resolution EP recordings at desired locations with patient's heart 10).

FIG. 4(a) shows one embodiment of EP mapping catheter 110 in a configuration where mapping electrode assembly 120 has not yet been deployed by the physician, and does not extend outwardly from tip 112 or outside slidable sheath 104. Handle or electrode deployment and control mechanism 102 remains outside the patient while the distal tip 112 of catheter is advanced towards the desired target inside or near patient's heart 10.

FIG. 4(b) shows the embodiment of EP mapping catheter 110 of FIG. 3(a) where mapping electrode assembly 120 is partially deployed such that 16 electrodes 122 on splines 126 are exposed at or near tip 112. (In the embodiment of EP mapping catheter 110 shown in FIGS. 4(a) through 4(b), mapping electrode assembly 120 comprises 8 splines 126, and each spline 126 has a total of 8 sensing or other electrodes 122 disposed or mounted thereon. Other numbers of splines and electrodes in catheter 110 are also contemplated, as described elsewhere herein.)

In FIG. 4(b), outer slidable sheath 108 has been withdrawn backwardly by the physician in the direction of handle 102 a distance D₁ from initial position W of FIG. 4(a) to position X of FIG. 4(b). As slidable sheath 104 is withdrawn from tip 112 towards position X, the initially distal-most portions of splines 126 of mapping electrode assembly 120 become exposed gradually. Representative but non-limiting examples of distance D₁ between W and X in FIG. 4(b) range between about 0.5 cm and about 2 cm.

In the configuration of partially deployed mapping electrode assembly 120 shown in FIG. 4(b) at distal portion 108 of catheter 110, a total of 16 electrodes 122, two on each spline, are exposed and available to take EP recordings. In the partially deployed configuration of FIG. 4(b), sufficient electrodes 122 are exposed, and electrodes 122 may be configured according to inter-electrode spacing and the size or surface area of the electrodes, to permit high-quality EP recordings to be taken, by way of non-limiting example, at or near a pulmonary vein (PV), in a manner similar to that obtained using a LASSO catheter as described above. Note that mapping electrode assembly 120 may also be deployed, and sheath 104 withdrawn a distance less than D₁, such that, for example, only the first 8 electrodes 112 mounted on or attached to splines 126 are exposed and available to make EP recordings.

Continuing to refer to FIG. 4(b), mapping electrode assembly 120 further comprises flexible (and in some embodiments extendible and/or elastic) tendons or chords 115 that connect adjoining splines 126. Tendons or chords 115 are configured to hold the ends of splines 126 in predetermined positions relative to one another as mapping electrode assembly 120 is progressively deployed. Tendons or chords 115 may be formed of any suitable biocompatible material, such as an elastic material, a wound, braided, stranded, twisted or thread-like material (such as KEVLAR or metal or metal alloy wires), a polymer, a metal or metal alloy, or a polymer- or otherwise biocompatible-material-coated metal, metal alloy, stranded, braided or twisted metal or metal alloy wires, polymeric fibers or threads, carbon fibers or the like, and may be attached or connected to splines 126 via tendon attachment points or structures 118 comprising a suitable adhesive such as epoxy, or may be crimped, swaged, stapled, or welded thereto at tendon or chord connection points or structure 118. Proximal portion 116 of catheter 110 shown in FIG. 4(b) includes external electrical connector 128, which permits electrical connections to be established between electrodes 122 of mapping electrode assembly 120 and the various modules of system 100, such as data acquisition device 140 and ablation module 150. Electrical conductors are provided within catheter 110 between distal and proximal portions 108 and 116 thereof such that signals sensed by electrodes 112 can be routed to from such electrodes 122 to connector 128 and thence system 100. The number of such electrical conductors included in catheter body 106 may be reduced (or effectively increased) by including suitable multiplexing electronic circuitry (e.g., a multiplexer ASIC) within catheter 110 (e.g., in handle 102, in catheter body 106, or near or at distal tip 112 in cap 111). Note that in some embodiments, catheter 110 includes one or more ablation electrodes or other devices configured to ablate or treat tissue from distal end 112, and may also include pacing electrodes. Sensing electrodes 122 may also be configured to serve as pacing electrodes. In some embodiments, catheter 110 includes navigation elements, coils, markers and/or electrodes so that the precise positions of the sensing, pacing and/or ablation electrodes inside the patient's heart 10 can be determined.

In some embodiments, splines 126 disclosed and described herein comprise a biocompatible shape memory alloy (e.g., nickel titanium, or Nitinol), and have been treated and configured during the process of manufacturing splines 126 and catheter 110 such that splines 126 will curl backwardly in the direction of proximal portion 116 of catheter as they are progressively exposed by the withdrawal of sheath 104 (or as spines 126 are advanced from distal end 112 of catheter 110, more about which is said below).

Nitinol is a metal alloy of nickel and titanium, where the two elements are typically present in roughly equal atomic percentages, e.g., Nitinol 55, Nitinol 60. The properties of the Nitinol or other suitable shape memory alloy employed in splines 126 are particular to the precise composition of the alloy used and its processing, and in some embodiments exhibit shape memory effect (SME) and superelasticity (SE; also called pseudoelasticity, PE). Nitinol is highly biocompatible, and has properties suitable for use in medical devices inserted or implanted within the human body. Due to Nitinol's unique properties, finds application in catheters, stents, and superelastic needles. In embodiments where the shape memory alloy selected for use in catheter 110 is Nitinol, tight compositional control of the Nitinol is required during the manufacturing process due to the high reactivity of titanium. By way of example, melting methods of the Nitinol employed to form splines 126 may include vacuum arc remelting (VAR) or vacuum induction melting (VIM). High vacuums may be required during a Nitinol spline manufacturing process. Alternatives to VAR and VIM include, but are not limited to, plasma arc melting, induction skull melting, and e-beam melting. Physical vapor deposition may also be employed. Some methods of working Nitinol for use in splines 126 include, but are not limited to, grinding, abrasive cutting, electrical discharge machining (EDM), and laser cutting. Heat treating of Nitinol employed in splines 126 can include varying aging time and temperature controls to obtain a desired Ni-rich phase and transformation temperature of splines 126, and thus control how much nickel resides in the resulting NiTi lattice. With respect to catheter 110 and splines 126 thereof, Nitinol is worked, treated and formed so that it will consistently and reliably behave and assume one or more of the various configurations shown and described herein as mapping electrode assembly 120 is progressively deployed from distal end 112 of catheter 110.

In alternative embodiments, splines 126 comprise a biocompatible material having shape memory characteristics and attributes, but are not formed of Nitinol or other shape memory alloys (or at least are not formed primarily or solely of one or more shape memory alloys). By way of non-limiting example, in such alternative embodiments splines 126 are formed of biocompatible shape memory materials such as shape-memory polymers, laminated 3D printed splines comprising shape memory materials, shape memory composites, and/or shape memory hybrids.

Referring now to FIG. 4(c), there is shown the embodiment of EP mapping catheter 110 of FIG. 4(a), where mapping electrode assembly 120 has been more fully deployed such that 32 electrodes 122 on eight splines 126 are exposed backwardly from tip 112. In FIG. 4(c), outer slidable sheath 108 has been withdrawn by the physician in the direction of handle 102 a distance D₂ from initial position W of FIG. 4(a) to position Y of FIG. 4(c). As slidable sheath 104 is withdrawn from position X to position Y, further portions of splines 126 of mapping electrode assembly 120 become exposed. Representative but non-limiting examples of distance D₂ between W and Y in FIG. 4(c) range between about 2 cm and about 10 cm. In the configuration of partially deployed mapping electrode assembly 120 shown in FIG. 4(c), a total of 32 electrodes 122, four on each spline, are exposed and available to take EP recordings. In the partially deployed configuration of FIG. 4(c), sufficient electrodes 122 are exposed, and electrodes 122 may be configured according to inter-electrode spacing and the size or surface area of the electrodes, to permit high resolution EP recordings to be taken in a patient's atrium or ventricle, in a manner similar to that obtained, for example, using a PENTARAY catheter as described above, or similar to the ADVISOR HD GRID mapping catheter manufactured by St. Jude.

Referring now to FIG. 4(d), there is shown the embodiment of EP mapping catheter 110 of FIG. 4(a) where mapping electrode assembly 120 has been fully deployed to form a basket catheter such that 64 electrodes 122 on eight splines 126 are exposed rearwardly from tip 112. In FIG. 4(d), outer slidable sheath 108 has been withdrawn backwardly by the physician in the direction of handle 102 a distance D₃ from initial position W of FIG. 4(a) to position Z of FIG. 4(d). As slidable sheath 104 is withdrawn from position Y to position Z, further portions of splines 126 of mapping electrode assembly 120 become exposed. Representative but non-limiting examples of distance D₄ between W and Z in FIG. 4(d) range between about 3 cm and about 20 cm. In the configuration of fully deployed mapping electrode assembly 120 shown in FIG. 4(d), a total of 64 electrodes 122, eight on each spline, are exposed and available to take EP recordings. In the fully deployed configuration of FIG. 4(d), sufficient electrodes 122 are exposed, and electrodes 122 may be configured according to inter-electrode spacing and the size or surface area of the electrodes, to permit medium resolution EP recordings to be taken in a patient's atrium or ventricle, in a manner somewhat similar, by way of non-limiting example, to that obtained using a Boston Scientific CONSTELLATION catheter (excepting, of course, the increased maneuverability and positionability of catheter 110).

Fully deployed mapping electrode assembly 120 of FIG. 4(d) further comprises and forms a basket having an interior open space 129 formed by fully expanded splines 126, which in some embodiments are spaced apart from one another along the circumference forming the basket at regular or fairly regular intervals. Moreover, also shown in FIG. 4(d) is opening 125, where no tendon or connector is disposed between two adjoining splines 126, which permits catheter body 106 and outer sheath 104 in distal portion 108 of catheter 110 to swing away from the longitudinal axis of, and partially outside, the basket, more about which is said below. This feature allows fully deployed mapping electrode assembly 120 to be positioned inside a patient's atrium or ventricle with improved accuracy and enhanced electrode coupling to the atrial or ventricular wall relative to that which can be achieved with a conventional basket catheter, and enables extra degrees of freedom, movement and positioning to be attained relative to a conventional prior art basket catheter.

Thus, and in reference to FIGS. 4(a) through 4(d), it will now be seen that in some embodiments mapping electrode assembly 120 of catheter 110 is capable of assuming different configurations while positioned within or near patient's heart 10 according to the particular application at hand. For example, EP recordings of the PVs, atria and ventricles at different spatial resolutions and in different locations within and near the heart 10 and PV16 can be made, all using the same catheter 110.

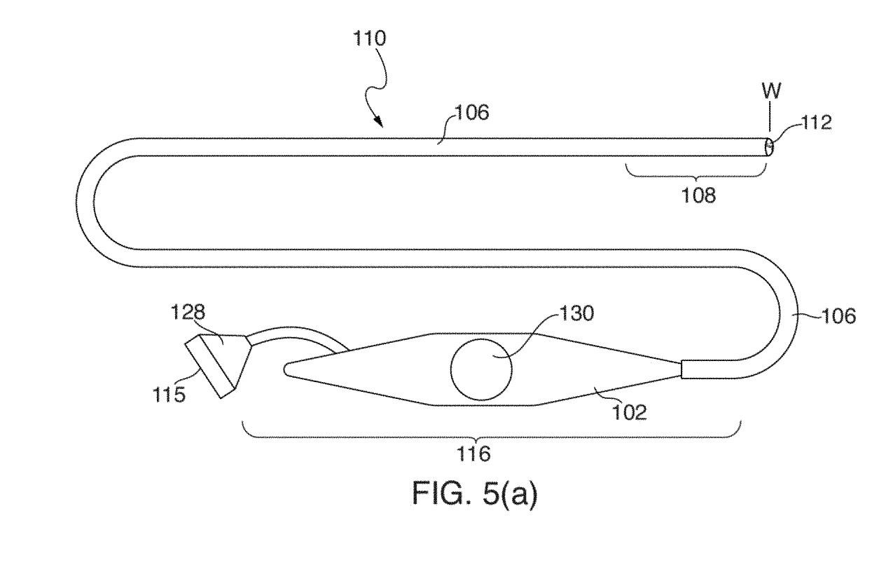

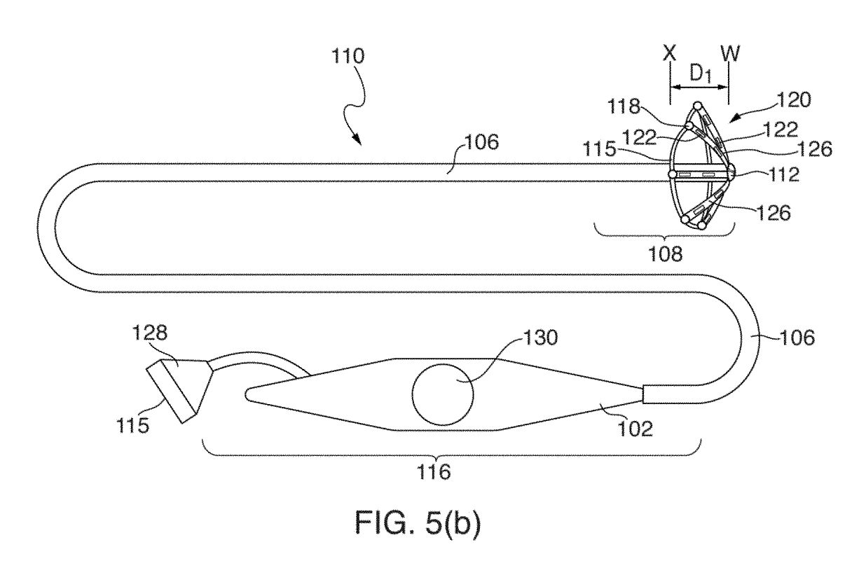

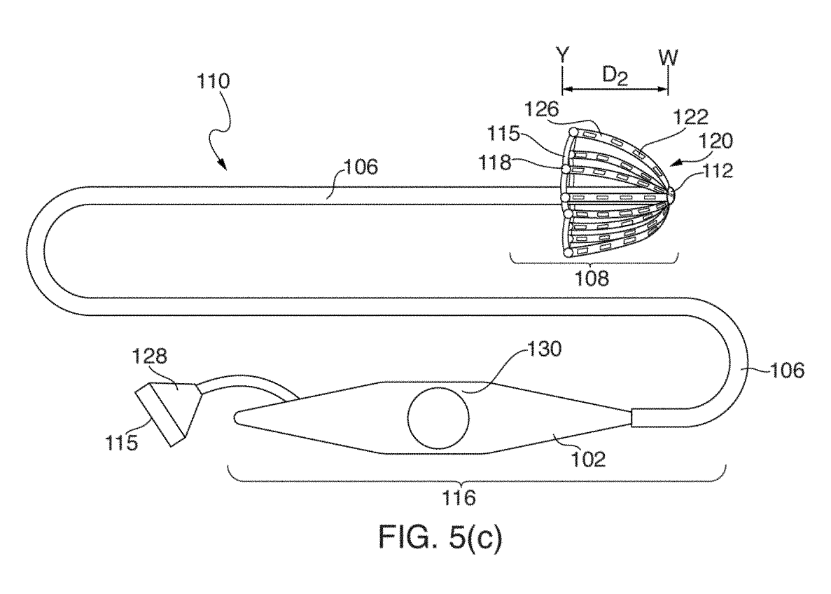

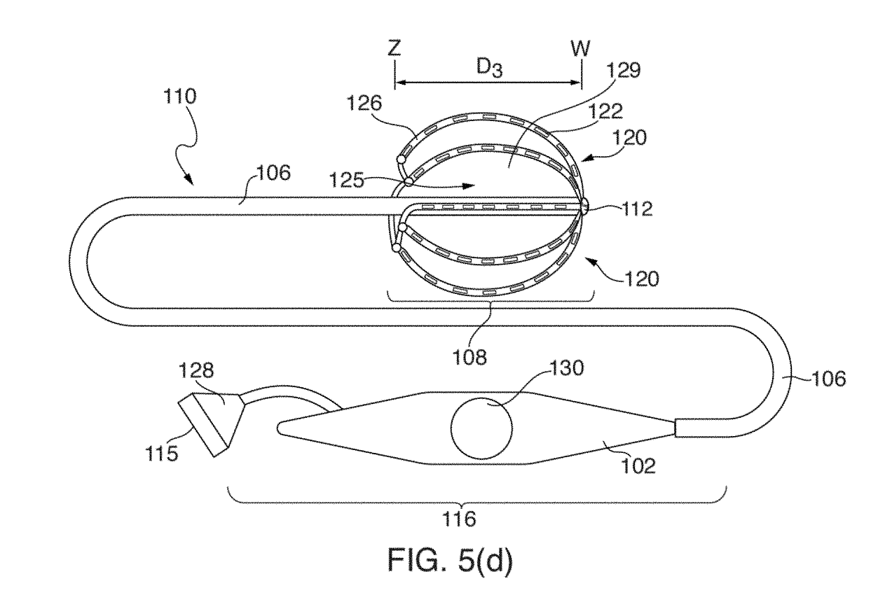

Referring now to FIGS. 5(a) through 5(d), there is shown another embodiment of EP mapping catheter 110 comprising mapping electrode assembly 120 located at the distal portion 108 of catheter 110, where no outer sheath 104 is provided, and where mapping electrode assembly 120 is instead deployed by pushing mapping electrode assembly 120 out of the distal end 112 of catheter 110 by advancing one or more wires, stylets, or other suitable pushing mechanisms in the distal direction of catheter 100 through the control and operation, by the physician, of deployment mechanism 130 located in handle 102. In FIGS. 5(a) through 5(d), once distal end 112 of EP mapping catheter 110 has been guided to a desired location within patient's heart 10, mapping electrode assembly 120 can be deployed in one or more configurations within patient's heart 10 according to the physician's objectives at hand (e.g., obtain EP recordings at or near the PV or inside atrium 12 or 14, and/or obtain low, medium or high resolution EP recordings at desired locations with patient's heart 10).

FIG. 5(a) shows one embodiment of EP mapping catheter 110 in a configuration where mapping electrode assembly 120 has not yet been deployed by the physician, and does not extend outwardly from tip 112.

FIG. 5(b) shows the embodiment of EP mapping catheter 110 of FIG. 5(a) where mapping electrode assembly 120 is partially deployed such that 16 electrodes 122 on splines 126 are exposed at or near tip 112. (In the embodiment of EP mapping catheter 110 shown in FIGS. 5(a) through 5(b), mapping electrode assembly 120 comprises 8 splines 126, and each spline 126 has a total of 8 sensing or other electrodes 122 disposed or mounted thereon. Other numbers of splines and electrodes in catheter 110 are also contemplated, as described elsewhere herein.)

In FIG. 5(b), EP mapping electrode assembly 120 has been advanced by the physician outside distal end 112 of catheter 110, and in the direction of handle 102 a distance D₁ from initial position W of FIG. 5(a) to position X of FIG. 5(b). As mapping electrode assembly 120 is pushed out of distal end 112 towards position X, the initially distal-most portions of splines 126 of mapping electrode assembly 120 become exposed gradually. Representative but non-limiting examples of distance D₁ between W and X in FIG. 5(b) range between about 0.5 cm and about 2 cm.

In the configuration of partially deployed mapping electrode assembly 120 shown in FIG. 5(b) at distal portion 108 of catheter 110, a total of 16 electrodes 122, two on each spline, are exposed and available to take EP recordings. In the partially deployed configuration of FIG. 5(b), sufficient electrodes 122 are exposed, and electrodes 122 may be configured according to inter-electrode spacing and the size or surface area of the electrodes, to permit high-quality EP recordings to be taken, by way of non-limiting example, at or near a pulmonary vein (PV), in a manner similar to that obtained using a LASSO catheter as described above. Note that mapping electrode assembly 120 may also be deployed such that, for example, only the first 8 electrodes 112 mounted on or attached to splines 126 are pushed out of the distal end of catheter 110 to make EP recordings.

Continuing to refer to FIG. 5(b), mapping electrode assembly 120 further comprises flexible (and in some embodiments extendible and/or elastic) tendons or chords 115 that connect adjoining splines 126. Tendons or chords 115 are configured to hold the ends of splines 126 in predetermined positions relative to one another as mapping electrode assembly 120 is progressively deployed. Tendons or chords 115 may be formed of any suitable biocompatible material, such as an elastic material, a wound, braided, stranded, twisted or thread-like material (such as KEVLAR or metal or metal alloy wires), a polymer, a metal or metal alloy, or a polymer- or otherwise biocompatible-material-coated metal, metal alloy, stranded, braided or twisted metal or metal alloy wires, polymeric fibers or threads, carbon fibers or the like, and may be attached or connected to splines 126 via tendon attachment points or structures 118 comprising a suitable adhesive such as epoxy, or may be crimped, swaged, stapled, or welded thereto at tendon or chord connection points or structure 118. Proximal portion 116 of catheter 110 shown in FIG. 5(b) includes external electrical connector 128, which permits electrical connections to be established between electrodes 122 of mapping electrode assembly 120 and the various modules of system 100, such as data acquisition device 140 and ablation module 150. Electrical conductors are provided within catheter 110 between distal and proximal portions 108 and 116 thereof such that signals sensed by electrodes 112 can be routed to from such electrodes 122 to connector 128 and thence system 100. The number of such electrical conductors included in catheter body 106 may be reduced (or effectively increased) by including suitable multiplexing electronic circuitry (e.g., a multiplexer ASIC) within catheter 110 (e.g., in handle 102, in catheter body 106, or near or at distal tip 112 in cap 111). Note that in some embodiments, catheter 110 includes one or more ablation electrodes or other devices configured to ablate or treat tissue from distal end 112, and may also include pacing electrodes. Sensing electrodes 122 may also be configured to serve as pacing electrodes. In some embodiments, catheter 110 includes navigation elements, coils, markers and/or electrodes so that the precise positions of the sensing, pacing and/or ablation electrodes inside the patient's heart 10 can be determined.

Referring now to FIG. 5(c), there is shown the embodiment of EP mapping catheter 110 of FIG. 5(a), where mapping electrode assembly 120 has now been more fully deployed such that 32 electrodes 122 on eight splines 126 are exposed backwardly from tip 112. In FIG. 5(c), mapping electrode assembly 120 has been pushed further out of distal tip 112 by the physician in the direction of handle 102 a distance D₂ from initial position W of FIG. 5(a) to position Y of FIG. 5(c). As slidable sheath 104 is withdrawn from position X to position Y, further portions of splines 126 of mapping electrode assembly 120 become exposed. Representative but non-limiting examples of distance D₂ between W and Y in FIG. 5(c) range between about 2 cm and about 10 cm. In the configuration of partially deployed mapping electrode assembly 120 shown in FIG. 5(c), a total of 32 electrodes 122, four on each spline, are exposed and available to take EP recordings. In the partially deployed configuration of FIG. 5(c), sufficient electrodes 122 are exposed, and electrodes 122 may be configured according to inter-electrode spacing and the size or surface area of the electrodes, to permit high resolution EP recordings to be taken in a patient's atrium or ventricle, in a manner similar to that obtained using, for example, a PENTARAY catheter as described above, or similar to the ADVISOR HD GRID mapping catheter manufactured by St. Jude.

Referring now to FIG. 5(d), there is shown the embodiment of EP mapping catheter 110 of FIG. 5(a) where mapping electrode assembly 120 has been fully deployed to form a basket catheter such that 64 electrodes 122 on eight splines 126 are exposed rearwardly from tip 112. In FIG. 5(d), mapping electrode assembly 120 has been fully advanced towards and then outside and backwardly from distal tip 112 of catheter 110 a distance D₃ by the physician through the action of deployment mechanism 130 located on handle 102 from initial position W of FIG. 5(a) to position Z of FIG. 5(d). As mapping electrode assembly 120 is pushed further out of distal end 112 of catheter 110, from position Y to position Z, further portions of splines 126 of mapping electrode assembly 120 become exposed. Representative but non-limiting examples of distance D₄ between W and Z in FIG. 5(d) range between about 3 cm and about 20 cm. In the configuration of fully deployed mapping electrode assembly 120 shown in FIG. 5(d), a total of 64 electrodes 122, eight on each spline, are exposed and available to take EP recordings. In the fully deployed configuration of FIG. 5(d), sufficient electrodes 122 are exposed, and electrodes 122 may be configured according to inter-electrode spacing and the size or surface area of the electrodes, to permit medium resolution EP recordings to be taken in a patient's atrium or ventricle, in a manner similar, by way of non-limiting example, to that obtained using a Boston Scientific CONSTELLATION catheter.

Fully deployed mapping electrode assembly 120 of FIG. 5(d) further comprises and forms a basket having an interior open space 129 formed by fully expanded splines 126, which in some embodiments are spaced apart from one another along the circumference forming the basket at regular or fairly regular intervals. Moreover, also shown in FIG. 5(d) is opening 125, where no tendon or connector is disposed between two adjoining splines 126. Similar to the embodiment of catheter 110 shown in FIGS. 3(a) through 3(d) and discussed above, opening 125 permits catheter body 106 of distal portion 108 of catheter 110 to swing away from the longitudinal axis of, and partially outside, the basket, more about which is said below. This feature allows fully deployed mapping electrode assembly 120 to be positioned inside a patient's atrium or ventricle with improved accuracy and enhanced electrode coupling to the atrial or ventricular wall relative to that which can be achieved with a conventional basket catheter, and enables extra degrees of freedom, movement and positioning to be attained relative to a conventional prior art basket catheter.

Thus, and in reference to FIGS. 5(a) through 5(d), it will now be seen that in some embodiments mapping electrode assembly 120 of catheter 110 is capable of assuming different configurations while positioned within or near patient's heart 10 according to the particular application at hand. For example, EP recordings of the PVs, atria and ventricles at varying spatial resolutions and in different locations within and near the heart 10 and PV16 can be made, all using the same catheter 110.

Referring now to FIGS. 4(a) through 5(d), electrodes 122 on splines 126 can be assigned electrode labels or addresses such as, by way of non-limiting example, A1 through H8. Catheter body 106 needs to be flexible so that it can be advanced through the patient's blood vessels towards the target site or location. Electrodes 122 are configured to sense electrical activity (e.g., activation signals, rotors, re-entry points, exit points, and the like) in tissue, such as heart tissue and pulmonary vein tissue. As described above, sensed signals provided by catheter 110 and electrodes 122 are processed by system 100 to assist the physician in identifying the specific site or sites where cardiac heart rhythm disorders or other pathologies originate or are manifested in heart, vein or other tissue. This information can then be used to determine an appropriate location for applying an appropriate therapy, such as ablation, to the identified sites, and also to navigate the one or more ablation or treatment electrodes to the identified sites. As discussed above, in some embodiments splines 126 are made of a shape memory alloy such as Nitinol. Other metals, metal alloys, combinations or laminations of metal or other materials such as KEVLAR, silicone, rubber, suitable polymers, may also be employed to form splines 126 to form resilient, pre-tensioned members (including shape-memory members) that are configured to bend and conform to the tissue surface with which they come into contact. In the embodiments illustrated in FIGS. 4(d) and 5(d), eight splines 126 form a basket structure. As discussed above, additional or fewer splines 126 can be employed in other embodiments. As illustrated in FIGS. 4(d) and 5(d), each spline 126 carries 8 mapping electrodes 122. In other embodiments, additional or fewer mapping electrodes 122 may be disposed on each spline 126.

While an arrangement of 64 mapping electrodes 122 is shown in FIGS. 4(d) and 5(d), mapping electrodes 122 in mapping electrode assembly 120 may be arranged in different numbers (more or fewer splines and/or more or fewer electrodes), on different structures, in different positions, or arranged at varying spacing along splines 126. In addition, in some embodiments multiple circular, fan-like or basket structures can be deployed in the same or different anatomical structures to simultaneously obtain signals from different anatomical structures or portions of tissue.

After electrodes 122 of catheter 110 have been deployed in the desired configuration, and positioned adjacent to a target anatomical structure (e.g., a pulmonary vein, the left atrium, the left ventricle, the right atrium, or the right ventricle of heart 10) whose electrical activity is to be measured, or which is to be treated (e.g., ablated), system 100 is configured to record electrical signals from each electrode 122 situated near the target anatomical structure. As described above, and as shown in FIGS. 4(a) through 5(d), mapping electrode assembly 120 can be deployed in myriad different configurations, where different spatial resolutions between electrodes are employed, and where different numbers of electrodes 122 are employed to sense electrical signals. For example, in the embodiments of catheter 110 shown in FIGS. 4(d) and 5(d), where mapping electrode assembly 120 is shown fully deployed, the spacing between electrodes can be twice that of the embodiments shown in FIGS. 4(c) and 5(c). Thus, in fully expanded configurations of FIGS. 4(d) and 5(d), catheter 110 can be configured to provide half the spatial resolution but cover a greater surface area than the higher spatial resolution configurations of FIGS. 4(c) and 5(c). In a fully deployed basket configuration, mapping electrode assembly 120 may vary in size from a small basket (capable of mapping a small, localized section of the cardiac chamber) to a large basket (capable of mapping most or all of a cardiac chamber). Utilizing a small basket structure may result in system 100 having to combine localized recordings together. Localized recordings may overlap one another, and therefore, to achieve a “global” representation of the cardiac chamber, it may be necessary to combine, or “stitch,” local recordings together.

The arrangement, size, spacing and location of electrodes 122 along a spline 126, in combination with the specific geometry of the targeted anatomical structure, may contribute to the ability (or inability) of electrodes 122 to be electrically coupled adequately to cellular tissue. Because splines 126 are flexible and bendable, they are configured to permit substantial conformance to and physical coupling to differently-shaped and configured anatomical regions. In at least some embodiments, and according to the inevitable particularities of the geometry of a given patient's heart or pulmonary vein, in many cases catheter 110 permits good coupling of most or all electrodes 122 to the patient's heart or vein tissue at or near the target site owing to the flexibility and shape memory of splines 126, the configurability and variable geometry mapping electrode assembly 120 is capable of assuming under the control of the physician, and the off-axis movement of the resulting electrode sensing array permitted by opening 125 of mapping electrode assembly 120 (more about which is said below).

Mapping electrode assembly 120 may also be employed to facilitate the assessment of entrainment, conduction velocity studies, and refractory periods in patient's heart 10. In some embodiments, mapping electrode assembly 120 further permits the simultaneous acquisition of longitudinal and circumferential signals along splines 126 for accurate 3-D mapping, and provides a flexible circular, fan-shaped, or basket geometry that is configured to conform to atrial or ventricular anatomy, and which permits greater accuracy in positioning and placement within patient's heart 10. Sixty-four electrodes A1 through H8 (or individual electrodes 122) can provide comprehensive, real-time 3-D information over a single heartbeat.

Continuing to refer to FIGS. 4(a) through 5(d), it will be seen that distances W-X (D₁), W-Y (D₂) and W-Z (D₃) correspond approximately to the respective lengths of mapping electrode assembly 120 that is exposed and available to sense electrical or other signals in a patient's internal organ (such as patient's heart 10). Moreover, the overall length of catheter 110 can be configured for applications in different types of patient's and applications, such as pediatric applications (where shorter overall lengths are preferred), applications in persons who have large frames (where longer overall lengths are preferred), gastric and esophageal applications (where lengths different from catheters configured for intra-cardiac applications are preferred), different access points for the catheter (e.g., femoral vein, femoral veins, internal jugular vein, subclavian vein, etc.). By way of non-limiting example, in intra-cardiac applications an overall length of catheter 110 between handle 102 and distal tip 112 can range between about 40 cm and about 200 cm, and in some embodiments the overall length is about 145 cm.

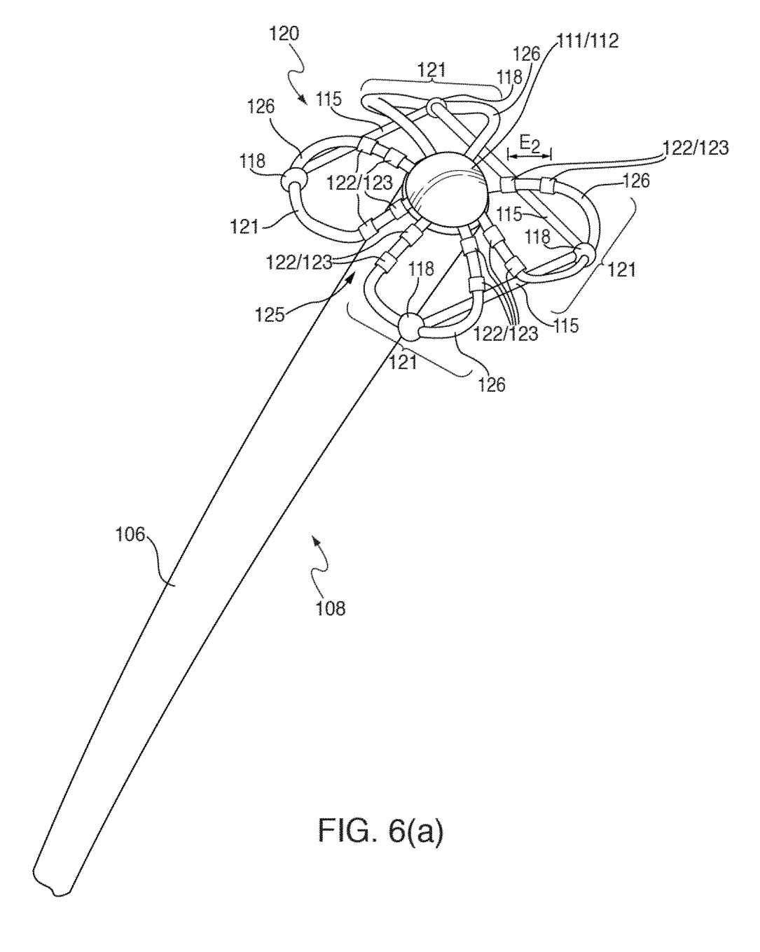

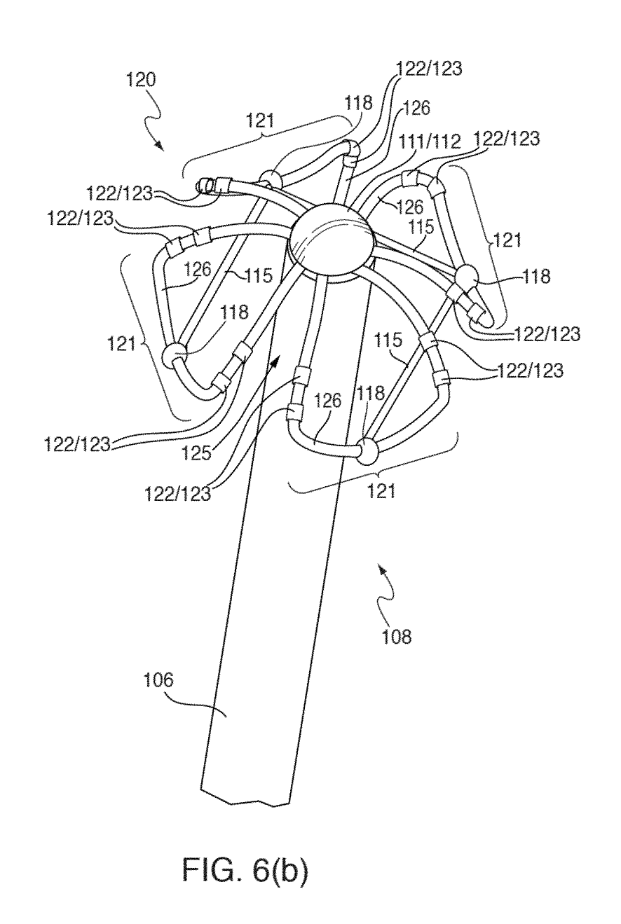

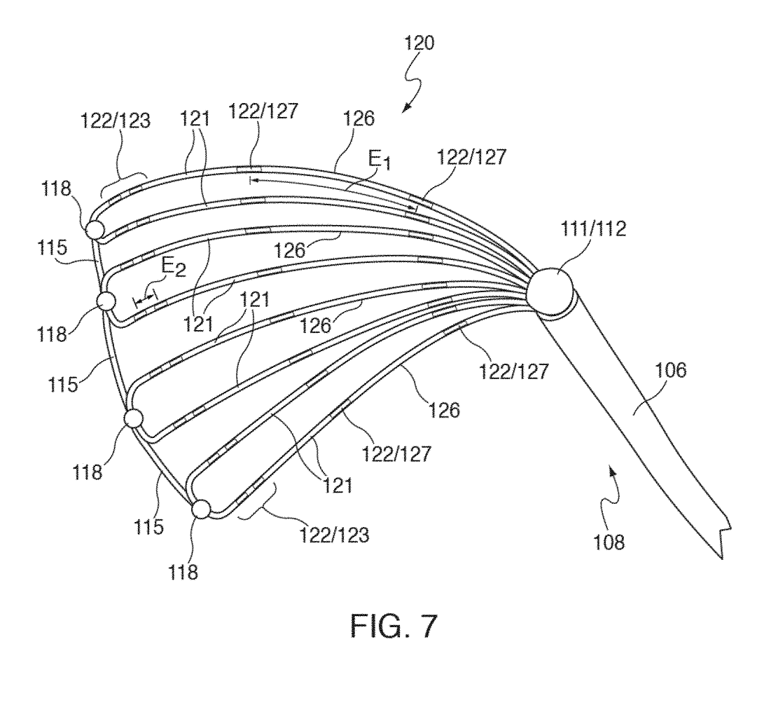

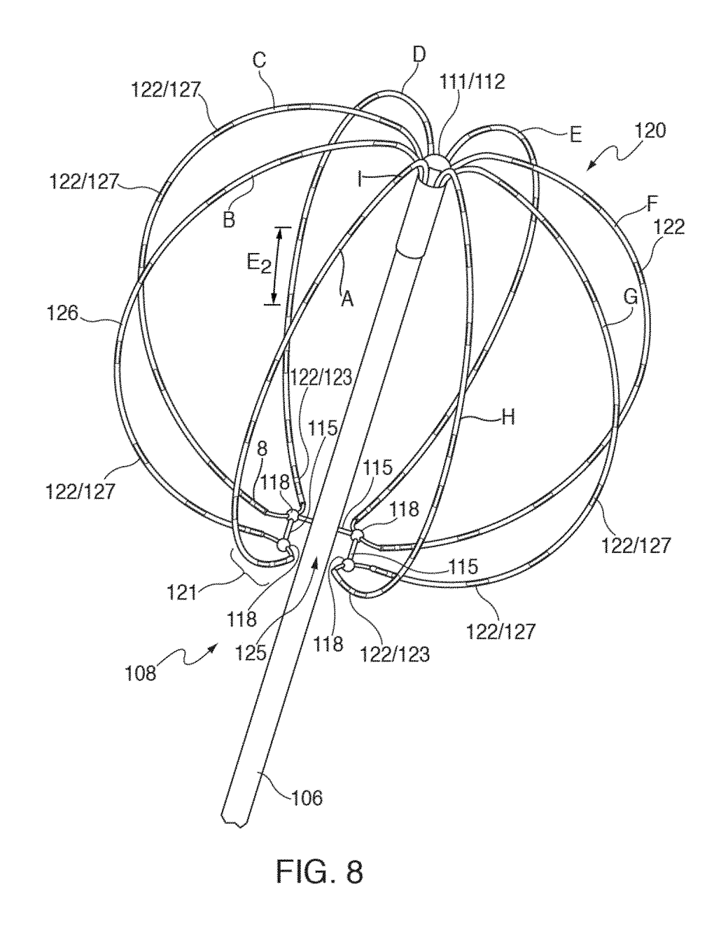

In respect of the terms “high resolution,” “high spatial resolution,” “medium resolution,” and “medium spatial resolution” as they are employed herein, note the following. In some embodiments, electrodes 122/127 are located along splines 126 at distances from one another ranging between about 1.2 cm to about 1.6 cm (see electrode spacing E₂ described below in connection with fan-shaped mapping electrode assembly 120 of FIG. 7). Closer electrode spacing of about 0.25 mm to about 2 mm along splines 126 may be employed for bipolar electrodes 122/123 used in a circular or lasso-like configuration (see electrode spacing E₁ described below in connection with mushroom-shaped mapping electrode assembly 120 of FIGS. 6(a) and 6(b)). In the mushroom-shaped configurations of mapping electrode assembly 120 shown in FIGS. 6(a) and 6(b), the finest and highest spatial resolution between electrodes is achieved by catheter 110 (which may be on the order of millimeters, e.g., about 0.25 mm to about 2 mm). In fan-shaped configurations of mapping electrode assembly 120 (such as that shown in FIG. 7), high spatial resolution is achieved by catheter 110, which in some embodiments, and depending on the manner and particular configuration in which mapping electrode assembly 120 is deployed and pressed and coupled against a patient's heart or other tissue, can range between nothing (splines touching) and about 2 cm. In basket configurations of mapping electrode assembly 120 (such as that shown in FIGS. 8-11 and 13), medium spatial resolution is achieved by catheter 110, which in some embodiments, and depending on the manner and particular configuration in which mapping electrode assembly 120 is deployed and pressed and coupled against a patient's heart or other tissue, can range between nothing (squished splines touching) to as much as 4 or 5 cm. Averaged spacings between electrodes in fine, high and medium resolution configurations that reduce the effects of touching splines can thus range, respectively, between about 0.25 mm and about 2 mm (fine spatial resolution), between about 0.25 cm and about 2 cm (high spatial resolution), and between about 1 cm and about 4 cm (medium spatial resolution).

Referring now to FIGS. 6(a) and 6(b), there is shown one embodiment of distal portion 108 of catheter 110 having mapping electrode assembly 120 initially deployed in a restricted or mushroom-shaped configuration, in two circular-shaped configurations and stages. In FIG. 6(a), mapping electrode assembly 120 is partially deployed such that only small pairs of bipolar electrodes 122/123 located on four arms 121 have been pushed outwardly from distal tip 112 beneath distal cap 111, for a total of 8 deployed sets of bipolar sensing electrodes. (Note that in some embodiments, bipolar pairs of electrodes 122/123 can be replaced with unipolar single electrodes 122, and vice-versa). In the embodiment shown in FIGS. 6(a) and 6(b), each of arms 121 comprises two splines 126 joined at tendon or chord connection point or structure 118 (although other numbers of splines 126 may be joined together or connected by structures 118). In FIGS. 6(a) and 6(b), tendons or chords 115 connect adjoining arms 121 comprising pairs of splines, and are likewise attached to tendon or chord connection points or structures 118. Tendons or chords 115 hold the ends of splines 126 in predetermined positions relative to one another as mapping electrode assembly 120 is progressively deployed.

As further shown in FIGS. 6(a) and 6(b), opening 125 disposed between two adjoining arms 121 located between arrow 125 in FIGS. 6(a) and 6(b) has no tendon or chord 115 disposed thereacross. Such a configuration permits distal portion 108 of catheter body 106 to swing or move outwardly away from the central longitudinal axis of deployed mapping electrode assembly 120 between two adjoining arms 121 through opening 125 (more about which is said below). This ability to partially decouple distal portion 108 of catheter 110 from mapping electrode assembly 120 permits more accurate, different and quicker placement, and better electrode coupling, of mapping electrode assembly near or at a target site than may be achieved with conventional basket catheters.

In the embodiment of catheter 110 shown in FIG. 6(a), splines 126, arms 121 and bipolar pairs of electrodes 122/123 extend but a small distance from distal end 112 of catheter 110. In one embodiment, a representative diameter of arms 121 in the configuration of distal portion 108 of catheter 110 shown in FIG. 6(a) is about 10 mm, or between about 8 mm and about 12 mm. Distal portion 108 of catheter 110 of FIG. 6(a) finds particularly efficacious application in EP mapping of small structures, such as, by way of non-limiting example, portions of tissue located at or near a pulmonary vein. As shown in FIG. 6(a), splines 126 and the shape memory material included therein may be configured and manufactured such that arms 121 of mapping electrode assembly 120 project mostly outwardly and only slightly downwardly when partially deployed in the circular fashion and configuration shown in FIG. 6(a). As shown in FIG. 6(a), spacing E₂ may be employed to separate each pair bipolar electrodes. In some embodiments, spacing E₂ ranges between about 0.5 mm and about 1 mm, or between about 0.25 mm and about 2 mm.

In the embodiment of catheter 110 shown in FIG. 6(b), splines 126, arms 121, and bipolar pairs of electrodes 122/123 extend a further distance from distal end 112 of catheter 110 than is shown in FIG. 6(a), and also finds particularly efficacious application in EP mapping of small structures, such as, by way of non-limiting example, portions of tissue located at or near a pulmonary vein. In one embodiment, a representative diameter of arms 121 in the configuration of distal portion 108 of catheter 110 shown in FIG. 6(b) is about 15 mm, or between about 12 mm and about 20 mm. Splines 126 and the shape memory material included therein may be configured such that arms 121 of mapping electrode assembly 120 project outwardly but further downwardly than is shown in FIG. 6(a) when partially deployed in the circular fashion and configuration shown in FIG. 6(b). In FIG. 6(b), in one embodiment, a representative but non-limiting length of tendon or chord 115 ranges between about 10 mm and about 15 mm. Consequently, the distance between adjoining splines 126 at the bottom portions thereof is set by the length of tendons or chords 115.

FIG. 7 shows one embodiment of distal portion 108 of catheter 110, where mapping electrode assembly 120 has been deployed in an intermediate fan- or paddle-shaped configuration extending further outwardly and backwardly from distal tip 112 with respect to the deployments of mapping electrode assemblies 120 shown in FIGS. 6(a) and 6(b). In FIG. 7, mapping electrode assembly 120 has been further partially deployed from distal end 112 into a fan-shaped configuration such that two further rows of larger unipolar electrodes 122/127 (with respect to the first row of smaller pairs of bipolar electrodes 122/123 shown in FIGS. 6(a) and 6(b)). Electrodes 122/123 and 122/127 are deployed on four arms 121 that have been pushed outwardly from distal tip 112 beneath distal cap 111. (Note that in some embodiments, bipolar pairs of electrodes 122/123 can be replaced with unipolar single electrodes 122, and vice-versa).

In the embodiment shown in FIG. 7, each of arms 121 comprises two splines 126 joined at tendon or chord connection point or structure 118 (although other numbers of splines 126 may be joined together or connected by structures 118). In FIG. 7, tendons or chords 115 connect three of adjoining arms 121 comprising pairs of splines 126, and are likewise attached to tendon or chord connection points or structures 118. Tendons or chords 115 hold the ends of splines 126 in predetermined positions relative to one another as mapping electrode assembly 120 is progressively deployed.

In FIG. 7, opening 125 shown in FIGS. 6(a) and 6(b) has become a large space located between the outer edges of fan-shaped mapping electrode assembly 120. The fan-shaped configuration of mapping electrode assembly 120 shown in FIG. 7 is a result of utilizing and implementing the shape memory effects of splines 126 and arms 121, and of the particular, customized, shape memory treating and manufacturing process that has been employed to make splines 126 and arms 121. That is, splines 126 and arms 121 are pre-bent, shaped, formed, and/or treated, and utilize one or more shape memory materials such as a shape memory metal alloy that will assume progressively different geometric configurations as mapping electrode assembly 120 is deployed ever further from distal end 120 of catheter 110.

As shown in FIG. 7, spacing E₁ may be employed to separate each row of electrodes 122/127 and 122/123 from one another. Note that electrode E₁ and E₂ may be varied according to the desired application. In some embodiments, spacing E₁ ranges between about 6 mm and about 20 mm, or between about 8 mm and about 18 mm, or between about 10 mm and about 15 mm. In some embodiments, inter-electrode spacing E₁ is varied in accordance with the maximum diameter obtained by mapping electrode assembly 120 in its fully deployed configuration, more about which is said below.

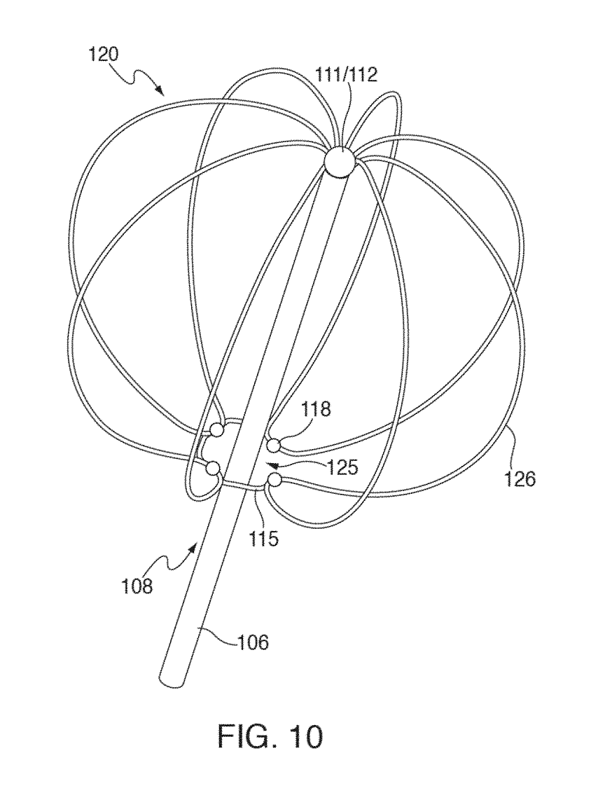

Referring now to FIG. 8, there is shown one embodiment of mapping electrode assembly 120 of FIGS. 6(a), 6(b) and 7 in a fully or nearly fully deployed basket configuration, where splines 126 have been pushed outwardly and backwardly fully from distal tip 112 from beneath cap 111. Again owing to the particular utilization and implementation of the shape memory effects inherent in splines 126 and arms 121, and the particular customized shape memory material or metal alloy treating and manufacturing process that has been employed to make splines 126 and arms 121, mapping electrode assembly 120, and arms 121 and splines 126 thereof, wrap around distal portion 108 of catheter 110 to form a distinct opening 125. Such a configuration of opening 125 permits distal portion 108 of catheter body 106 to swing or move outwardly away from a central longitudinal axis of deployed mapping electrode assembly 120 between two adjoining arms 121 through opening 125 (more about which is said below in connection with FIGS. 10 and 10(a)). In some embodiments, strong bending forces and characteristics may be employed in the shape memory materials forming the portions of splines 126 located near distal tip 112, while relatively weaker bending forces and characteristics are employed in the shape memory materials forming more centrally-located portions of splines 126 located in the equatorial regions of the resulting basket catheter. Employing disparate bending forces along splines 126 permits mapping electrode assembly 120 to switch from the fan-shaped configuration shown in FIG. 7 to the basket configuration shown in FIG. 8

In some embodiments, the resulting basket structure may have a diameter ranging between about 20 mm and about 200 mm, between about 30 mm and about 100 mm in diameter, between about 40 m and about 80 mm in diameter, and/or between about 50 mm and about 70 mm in diameter. In still other embodiments, the resulting basket structure is smaller or larger (e.g., less than 20 mm in diameter or greater than 200 mm in diameter). Basket diameters of about 50 mm, about 60 mm, and about 70 mm are also contemplated in the resulting basket structure. As discussed above, inter-electrode spacing E₁ may also be varied according to the resulting basket diameter. For example, in a 50 mm diameter embodiment, inter-electrode spacing E₁ may be about 10 mm, in a 60 mm diameter embodiment, inter-electrode spacing E₁ may be about 13 mm, and in a 70 mm diameter embodiment may be about 15 mm.

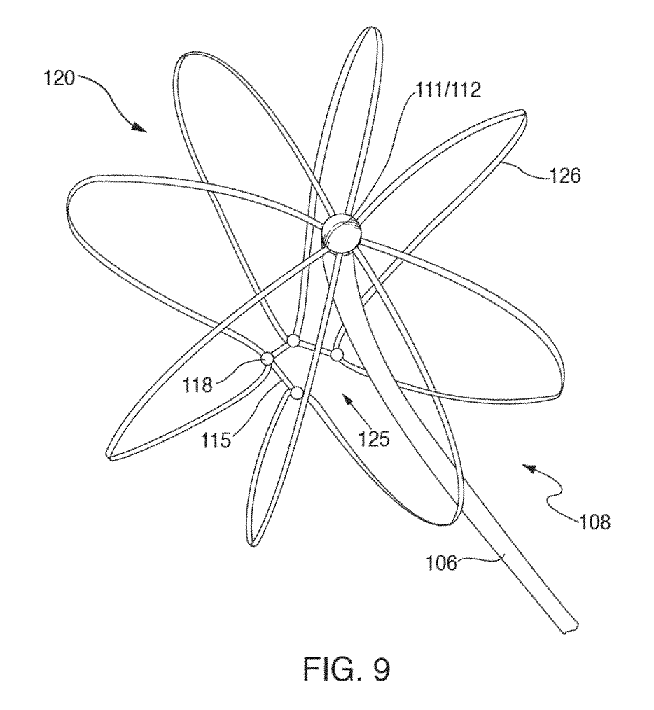

FIGS. 9 and 10 show front and side perspective views according to one embodiment of fully deployed mapping electrode assembly 120 of FIG. 8. For simplicity, electrodes are not shown on splines 126 mapping electrode assembly 120 of FIGS. 9 and 10. FIGS. 9 and 10 shows opening 125 through which catheter body 106 may move or swing away from central and bottom (or distally disposed) portions of mapping electrode assembly 120.

FIG. 11 shows one embodiment of distal portion 108 of catheter 110 where mapping electrode assembly 120 is in a fully deployed configuration, and where splines 126 have been pushed outwardly and backwardly fully from distal tip 112 from beneath cap 111. For simplicity, and as in FIGS. 9 and 10, electrodes are not shown on splines 126 mapping electrode assembly 120 of FIG. 11. As shown in FIG. 11, the basket formed by fully deployed mapping electrode assembly 120 has a first imaginary central longitudinal axis A-A′ associated therewith, around which splines 126 are evenly or fairly evenly arranged, opened and deployed. Also shown in FIG. 9 is a second imaginary axis B-B′ associated with portions of catheter body 106 located proximally from distal tip 112. The first and second imaginary axes A-A′ and B-B′ of FIG. 9 intersect one another at an angle θ. Opening 125 permits more proximally-located portions of catheter body 106 to be swung and moved outwardly away from central longitudinal axis A-A′ of the basket formed by fully deployed mapping electrode assembly 120 for alignment with axis B-B′. Being able to partially decouple catheter body 106 of catheter 110 from mapping electrode assembly 120 permits more accurate, different and quicker placement, and superior electrode coupling, of mapping electrode assembly 120 near or at a target site inside or near patient's heart 10 than may be achieved with a conventional basket catheter.

As further shown in FIG. 11, portions of catheter body 106 located just proximally from distal tip 112 may be configured such that tip 112 can be bent at location 119 and then steered in a desired direction by the physician. Such bending at location 119 of catheter body 106 and steering of tip 112 may be accomplished using a pull wire or stylet disposed inside catheter body 106, as is well known in the art.

The combination of a mapping electrode assembly 120 that can be decoupled from catheter body 106 and the ability to steer or bend tip 112 in catheter 110 results, relative to prior art EP mapping catheters, in substantial improvement of mapping electrode assembly 120 being placed in optimum EP mapping positions, and electrodes having optimum coupling to tissue, inside patient's heart 10. These desirable results are illustrated in FIG. 12, where one embodiment of football-shaped mapping electrode assembly 120 is shown fully deployed and electrically coupled inside patient's left atrium 14. In the example shown in FIG. 12, once fully deployed inside atrium 14, the combination of a bendable or steerable tip 112 and the decoupling mechanism enabled by opening 125 permits all or most portions of mapping electrode assembly 120 to be electrically coupled to the walls of atrium 14, including portions of the atrial walls that are actually located behind and to the left of the entry point of distal end 112. This may be accomplished, for example, by the physician pulling backwardly on catheter body 106 once electrode assembly 120 has been fully deployed in atrium 14. Given the steep entry angle of distal tip 112 into patient's atrium 14 via the foramen ovalen and trans-septal puncture, such optimal positioning and electrode coupling cannot be achieved using a conventional basket catheter (as illustrated in FIG. 13, where it is shown that a conventional basket catheter cannot be positioned, at least not without great difficulty, leftwardly from the atrial entry point of distal tip 112).

In other embodiments, catheter 110 is a basket catheter

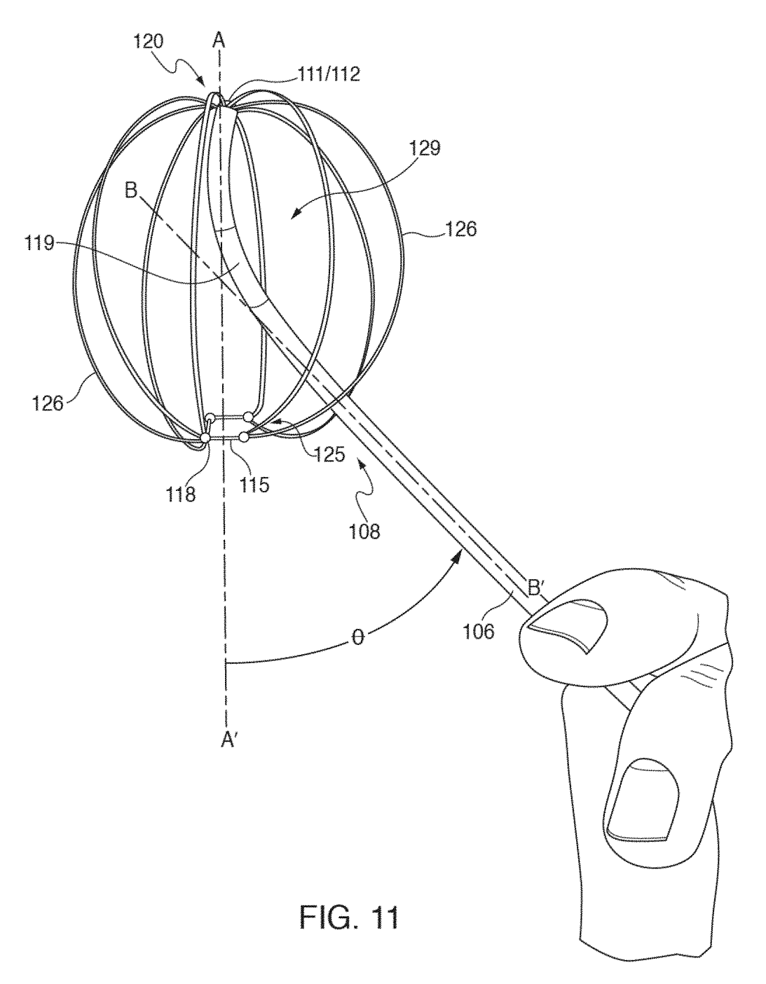

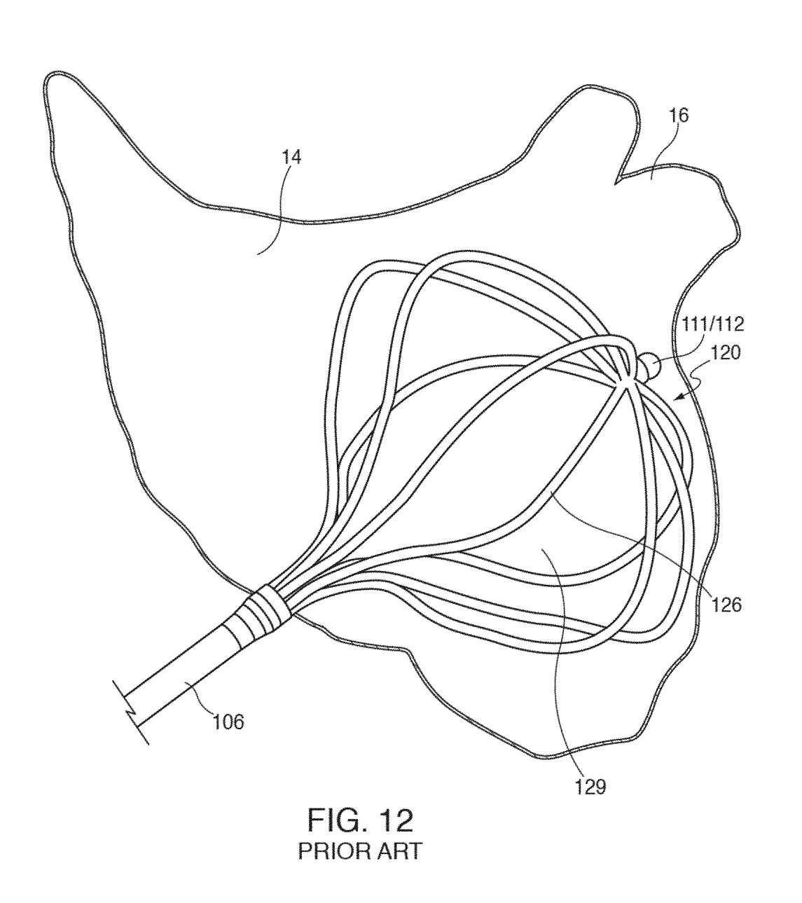

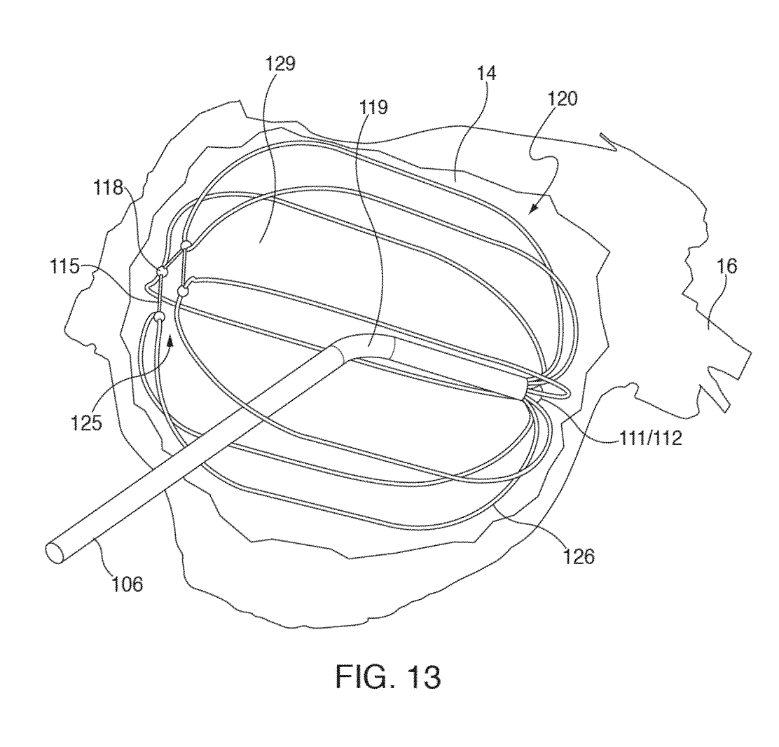

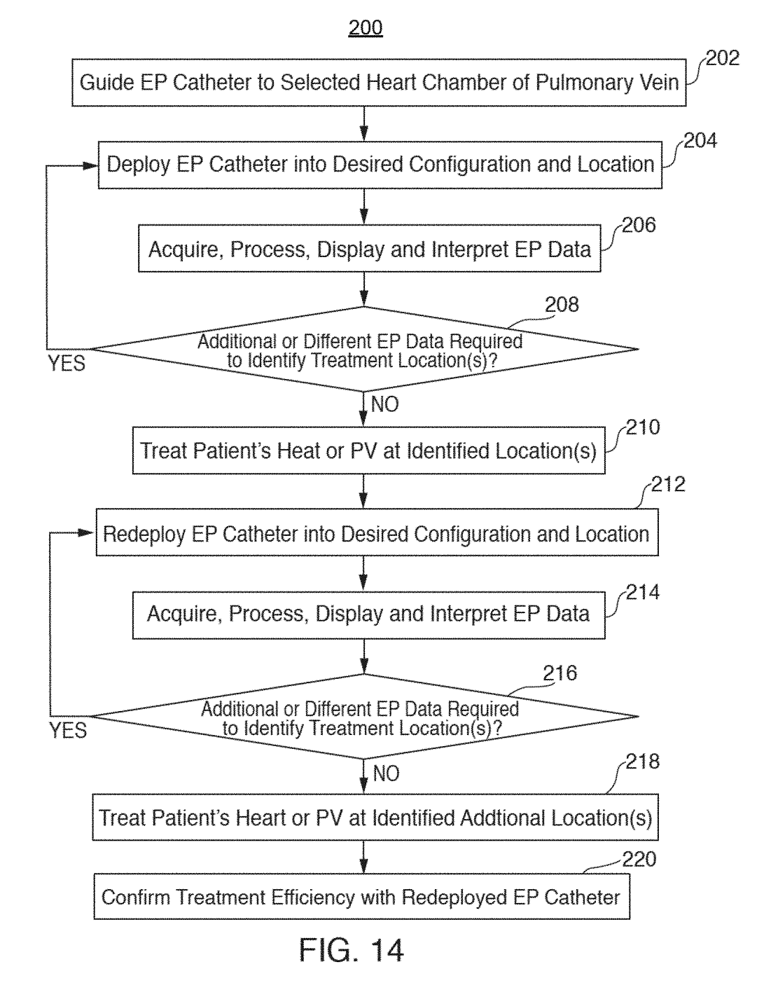

FIG. 14 shows one method 200 of using the configurable multi-application electrophysiological mapping catheter described above and shown in FIGS. 2 through 12. At step 202, configurable multi-application EP mapping catheter 110 is guided to a selected or desired site within or near patient 10's heart 10 (e.g., to the right or left atrium, or one of the pulmonary veins or arteries)). In some embodiments, imaging and/or navigation system 60 is employed help guide catheter 110 to the site. At step 203, and after being guided to the desired or selected site, configurable multi-application EP mapping catheter 110 is deployed into a desired electrode configuration (e.g., the mushroom-shaped electrode configuration of FIGS. 6(a) or 6(b), the fan-shaped electrode configuration of FIG. 7, or the basket-shaped electrode configuration of FIG. 8). EP data are then acquired, processed, displayed at step 206 using system 100, which are then interpreted by the physician or other health care professional. At step 208, the physician or other health care professional determines whether additional or different EP data are required to identify treatment locations within or near patient's heart 10. If so, steps 204 and 206 are repeated. If not, at step 210 patient's heart 10 is treated at the identified locations by, for example, ablating heart or pulmonary vein or artery tissue at the desired location identified in step 208. At step 212, configurable multi-application EP mapping catheter 110 is redeployed into a desired electrode configuration at a desired step, and at step 214 EP data are once again acquired, processed, and displayed using system 100. The results obtained in step 214 are then interpreted by the physician or other health care professional. At step 216, the physician or other health care professional determines whether additional or different EP data are required to identify additional treatment locations within or near patient's heart 10. If so, steps 212 and 214 are repeated. If not, at step 218 patient's heart 10 is treated at the additional identified locations by, for example, ablating heart or pulmonary vein or artery tissue at the desired location identified in step 216. At step 220, the efficacy and success of the treatment and surgery can be confirmed by redeploying configurable multi-application EP mapping catheter 110 to a desired site, and acquiring, processing, displaying and interpreting EP data.

Provided now are some illustrative details regarding the composition, materials, and manufacture of some embodiments of catheter 110.

Depth markers on proximal portion 116 of lead body 106 and/or on outer sheath 104 may be used by a physician to gauge the extent to distal tip 112 of catheter 110 has been inserted inside the patient. By way of example, depth markers may be formed of polyethylene heat shrink, or printed on lead body 15 using medical grade ink.

An electrically insulative material may be employed inside catheter body 106 to protect electrical conductors disposed within catheter body 106 from the corrosive effects presented by body fluids, and may be formed of a biocompatible material such as a suitable polyurethane, silastic compound, a fluoro-copolymer such as fluorinated ethylene propylene (FEP) or TEFLON 100™, nylon, or any other suitable electrically insulative material.

Outer sheath 104 and portions of catheter body 106 may comprise a biocompatible material such as polyethylene, or any other suitable polymer or polymeric compound such as PEBAX.

Catheter 110, catheter body 106, and outer sheath 104 (if used) may be configured to have lengths appropriate for pediatric use, use in persons having different body sizes, or implantation through different entry points such as the left or right subclavian vein, the internal jugular vein, or the right or left femoral veins. Additionally, catheter body 106 and catheter 110 may be configured to have lengths appropriate for implantation in the right atrium, the left atrium, the right ventricle, and/or the left ventricle.

In some embodiments, electrical conductors disposed within lead body 106, and that are operably attached to electrodes 122 and external connector 115, comprise one or more suitable flexible electrically conductive materials such as metal or metal alloy wires, or stranded, wound, braided and/or twisted metal or metal alloy wires that are capable of reliably conducting electrical current after having been subjected to numerous, repeated bending and torquing stresses. Such conductors may be formed, by way of non-limiting example, of wires comprising a nickel-titanium alloy such as NITINOL™, stainless steel, platinum, gold, silver, palladium, other noble metals, and other alloys or metals suitable for use in the human body.

In some embodiments, catheter body 106 has a diameter ranging between about 2 French and about 10 French, or between about 3 French and about 8 French, or between about 4 French and about 6 French. Other diameters of catheter body 106 are also contemplated. In addition, catheter 110 may have incorporated therein tissue ablation mechanisms and/or components, and/or force sensing mechanisms, such as those described in the aforementioned '924 patent application.

In one embodiment electrode assembly 120 is configured to be controllably deployed and advanced from distal tip 112 of catheter 110 by a user operating electrode deployment and control mechanism 102 into any two or more of the following configurations: (a) a first initial deployment configuration suitable for pulmonary vein isolation (PV) EP mapping (see, e.g., FIGS. 4(b), 5(b), 6(a), and 6(b)); (b) a second intermediate deployment fan or paddle configuration suitable for high-resolution EP mapping (see, e.g., FIGS. 4(c), 5(c), and 7); and (c) a third fully or nearly fully deployed basket configuration suitable for medium-resolution EP mapping, the basket configuration having imaginary central longitudinal axis A-A′associated therewith when the basket is deployed in an unobstructed and unconfined space (see, e.g., FIGS. 4(d), 5(d), 8, 9, 10, 11, and 13), and further wherein: (i) in the first configuration electrode mapping assembly 120 is deployed by the user a first distance from distal portion 108 of catheter body 106 (see, e.g., distance D₁ of FIGS. 4(b) and 5(b)); (ii) in the second configuration electrode mapping assembly 120 is deployed by the user a second distance from distal portion 108 of catheter body 106 (see, e.g., distance D₂ of FIGS. 4(c) and 5(c)); and (iii) in the third configuration electrode mapping assembly 120 is deployed by the user a third distance from distal portion 108 of catheter body 106 (see, e.g., distance D₃ of FIGS. 4(d) and 5(d)), and further wherein the first distance is less than the second distance, the second distance is less than the third distance, opening 125 is located between at least portions of two adjoining splines in electrode mapping assembly 120, no chord or tendon is located within at least portions of opening 125 such that portions of catheter body 108 located proximally from distal tip 112 can be moved by a user away from longitudinal axis A-A′ of the basket in a direction of opening 125 (see, e.g., FIG. 11). In such an embodiment, corresponding methods can comprise two or more of: (1) deploying electrode mapping assembly 120 into the first configuration inside or near patient's heart 10; (2) deploying electrode mapping assembly 120 into the second configuration inside or near patient's heart 10, and (3) deploying electrode mapping assembly 120 into the third configuration inside or near patient's heart 10.

In another embodiment electrode assembly 120 is configured to be controllably deployed and advanced from distal tip 112 of catheter 110 by a user operating electrode deployment and control mechanism 102 into a basket configuration, the basket configuration having an imaginary central longitudinal axis A-A′ associated therewith when the basket is deployed in an unobstructed and unconfined space, and further wherein opening 125 is located between at least portions of two adjoining splines in electrode mapping assembly 120, no chord or tendon 115 is located within at least portions of opening 125 such that portions of catheter body 106 located proximally from distal tip 112 can be moved by a user away from longitudinal axis A-A′ of the basket in a direction of opening 125. In such an embodiment, corresponding methods can comprise deploying the electrode mapping assembly into the basket configuration inside or near the patient's heart.

In yet another embodiment electrode assembly 120 is further configured to be controllably deployed and advanced from distal tip 112 of catheter 110 by a user operating electrode deployment and control mechanism 102 into the following configurations: (a) a first circular, semi-circular, oval, elliptical, or lasso-like configuration suitable for pulmonary vein isolation (PV) EP mapping (see, e.g., FIGS. 4(b), 5(b), 6(a), and 6(b)); and (b) a second basket configuration, the basket having imaginary central longitudinal axis A-A′ associated therewith when the basket is deployed in an unobstructed and unconfined space (see, e.g., FIGS. 4(d), 5(d), 8, 9, 10, 11, and 13), and further wherein: (i) in the first configuration electrode mapping assembly 120 is deployed by the user a first distance from distal portion 108 of the catheter body 106 (see, e.g., distance D₁ of FIGS. 4(b) and 5(b)), and (ii) in the second configuration electrode mapping assembly 120 is deployed by the user a second distance from distal portion 108 of catheter body 106 (see, e.g., distance D₃ of FIGS. 4(d) and 5(d)); and further wherein the first distance is less than the second distance, opening 125 is located between at least portions of two adjoining splines in electrode mapping assembly 120, no chord or tendon 115 is located within at least portions of opening 125 such that portions of catheter body 106 located proximally from distal tip 112 can be moved by a user away from longitudinal axis A-A′ of the basket in a direction of opening 125. In such an embodiment, corresponding methods can comprise at least one of: (1) deploying the electrode mapping assembly into the first configuration inside or near the patient's heart, and (2) deploying the electrode mapping assembly into the second configuration inside or near the patient's heart.

In still another embodiment electrode assembly 120 is configured to be controllably deployed and advanced from distal tip 112 of catheter 110 by a user operating electrode deployment and control mechanism 102 into any two or more of the following configurations: (a) a first fan-shaped configuration of mapping electrode assembly 120 wherein electrodes mounted on or attached to central portions of adjoining spines are separated from one another by distances ranging between about 0.25 cm and about 2 cm such that mapping electrode assembly 120 is configured to provide high spatial resolution EP data; and (b) a second basket configuration of mapping electrode assembly 120 wherein electrodes mounted on or attached to central portions of adjoining spines are separated from one another by distances ranging between about 1 cm and about 4 cm such that mapping electrode assembly 120 is configured to provide medium spatial resolution EP data, the basket configuration having imaginary central longitudinal axis A-A′ associated therewith when the basket is deployed in an unobstructed and unconfined space, and further wherein: (i) in the first configuration electrode mapping assembly 120 is deployed by the user a first distance from distal portion 108 of catheter body 106; (ii) in the second configuration electrode mapping assembly 120 is deployed by the user a second distance from distal portion 108 of catheter body 106; and further wherein the first distance is less than the second distance, opening 125 is located between at least portions of two adjoining splines in electrode mapping assembly 120, no chord or tendon 115 is located within at least portions of opening 125 such that portions of catheter body 106 located proximally from distal tip 112 can be moved by a user away from longitudinal axis A-A′ of the basket in a direction of opening 125. In such an embodiment, corresponding methods can comprise at least one of: (1) deploying electrode mapping assembly 120 into the first configuration inside or near patient's heart 10, and (2) deploying electrode mapping assembly 120 into the second configuration inside or near patient's heart 10.

In yet a further embodiment, EP mapping catheter 120 comprises elongated catheter body 106 comprising proximal portion 116, distal portion 108, and distal tip 112, electrode deployment and control mechanism 102 located near or at proximal portion 116 of catheter body 108, deployable electrode mapping assembly 120 operably connected to electrode deployment and control mechanism 102, electrode mapping assembly 120 comprising a plurality of electrodes 122/123/127 and a plurality of splines 126, each spline 126 having a proximal end and a distal end, electrodes 122/123/127 being mounted on or connected to at least some of splines 126, at least some of splines 126 comprising a shape memory material, at least the distal end of each spline 126 being configured to bend or be bent backwardly from distal tip 112 towards more proximal portions of catheter body 106 as the plurality of splines 126 is deployed from or near distal tip 112, wherein at least major portions of electrode mapping assembly 120 are configured to fit within distal portion 108 of catheter body 106 when electrode assembly 120 is in an undeployed configuration, electrode assembly 120 further being configured to be controllably deployed and advanced from distal tip 112 of catheter 110 by a user operating electrode deployment and control mechanism 102 into at least one of the following configurations: (a) a first circular, semi-circular, oval, elliptical, or lasso-like configuration suitable for pulmonary vein isolation (PV) EP mapping; (b) a second fan-shaped configuration of the mapping electrode assembly suitable for acquiring high-resolution EP data; and (c) a third basket configuration suitable for acquiring medium-resolution EP data. In such embodiments, opening 125 between splines 126 may—or may not—be included or provided in catheters 110 described herein. Methods of deploying and using catheter 110 according to such embodiments are also contemplated, as are catheters 110 capable of assuming only one of the aforementioned three configurations (e.g., circular, fan-shaped, and basket configurations).

In still further embodiments, any of the above- or below-described catheters 110 and corresponding methods can be modified such that there is no opening 125 located between adjoining splines 126 where portions of catheter body 106 located proximally from distal tip 112 can be moved by a user away from the longitudinal axis A-A′ of the basket through such opening 125. In such embodiments, movement of catheter body 106 outside proximally-located portions of a fully deployed or nearly fully-deployed basket-shaped mapping electrode assembly 120 may not be possible owing to the presence of chords or tendons 115 in the path of catheter body 106.

The foregoing embodiments may further comprise one or more of: catheter 110 being configured to permit portions of catheter body 106 located proximally from distal tip 112 to be moved by the user away from longitudinal axis A-A′ of the basket in the direction of and through opening 125; catheter 110 being configured to permit portions of catheter body 106 located proximally from distal tip 112 to be moved by the user away from longitudinal axis A-A′ of the basket in the direction of and outside opening 125; distal tip 112 of catheter 110 being configured to be steerable or bent by the user; outer slidable sheath 104 being configured to permit deployment of electrode mapping assembly 120 from distal tip 112 of the catheter; outer slidable sheath 104 being steerable or having a tip thereof that is steerable; a steerable sheath 104 comprising a steerable distal end; electrode mapping assembly 120 comprising between 4 splines and 12 splines 126; each spline 126 having attached thereto, mounted thereon or formed therein between 1 and 16 electrodes 122/123/127; distal ends of adjoining splines 126 forming pairs of splines 126 that are joined or connected to one another; one or more navigation elements, navigation coils, navigation markers or navigation electrodes; a shape memory material comprising one or more of Nitinol, a shape memory metal, a shape memory alloy, a shape memory polymer, a shape memory composite, or a shape memory hybrid; at least one spline 126 in electrode mapping assembly 120 comprising laminated materials; mapping electrode assembly 120 being deployed by pushing mapping electrode assembly 120 out of distal end 112 of catheter 110 using the electrode deployment and control mechanism; a tissue ablation mechanism located at or near distal tip 112 of catheter 110; spatial resolution provided by electrodes 122/123/127 in electrode mapping assembly 120 and an associated spacing between splines 126 changing in accordance with the first, second and third configurations thereof; a diameter of arms 121 of electrode mapping assembly 120 ranging between about 6 mm and about 14 mm when electrode mapping assembly 120 is deployed in the first configuration; a diameter of arms 121 of electrode mapping assembly 120 ranging between about 6 mm and about 14 mm when electrode mapping assembly 120 is deployed in the first configuration; a diameter of arms 121 of electrode mapping assembly 120 ranging between about 10 mm and about 20 mm when electrode mapping assembly 120 is deployed in the first configuration; a length of each tendon or chord 115 ranging between about 6 mm and about 20 mm; electrodes 122/123/127 being one or more of unipolar electrodes and bipolar electrodes; spacing between adjoining electrodes 122/123/127 located on the same spline 126 ranging between about 0.5 mm and about 1 mm, between about 0.25 mm and about 2 mm, between about 6 mm and about 20 mm, between about 8 mm and about 18 mm, or between about 10 mm and about 15 mm; the third basket structure having an outer diameter ranging between about 20 mm and about 200 mm, between about 30 mm and about 100 mm in diameter, between about 40 mm and about 80 mm in diameter, or between about 50 mm and about 70 mm, or is about 50 mm, about 60 mm or about 70 mm.

The foregoing embodiments may further comprise one or more of: distal tip 112 of catheter 110 being configured to be steerable or bent by the user, and the user bends or steers distal tip 112 of catheter 110 inside or near patient's heart 10; acquiring EP signals from the patient using electrodes 122/123/127 in deployed electrode mapping assembly 120; processing the acquired EP signals so that the signals may be interpreted by the user; redeploying electrode mapping assembly 120 into a different configuration or location within or near patient's heart 10 based upon results provided by the processed EP signals; changing the configuration of electrode mapping assembly 120 from one of the first, second and third configurations to a different configuration; deploying mapping electrode assembly 120 by pushing mapping electrode assembly 120 out of the distal end 112 of catheter 110 using electrode deployment and control mechanism 102; ablating tissue at a location in or near the patient's heart 10, the location being identified using the processed EP signals.

It will now become apparent to those skilled in the art that configurable multi-application EP mapping catheter 110 provides a significant advantage to physicians and patients alike, in that only a single EP mapping catheter need be employed to carry out all the steps of method 200 illustrated in FIG. 14, and that multiple EPO catheters need not be inserted within and withdrawn from the patient's heart and vasculature to obtain EP mapping data and information required to inform and guide the treatment process. It will now be seen that the various systems, devices, components and methods disclosed and described herein are capable of detecting with considerable accuracy and precision the locations of the sources of cardiac rhythm disorders in and near a patient's heart.

The various systems, devices, components and methods described and disclosed herein may also be adapted and configured for use in EP mapping and/or neurological sensing and mapping applications other than those involving the interior of a patient's heart or the pulmonary veins or arteries. These alternative applications include, but are not limited to, EP mapping and diagnosis, or other forms, means or methods of electrically sensing, a patient's stomach, colon, esophagus, veins, arteries, aorta, or any other suitable portion of a patient's body such as a patient's brain. The various embodiments further include within their scope methods of implanting, using and making the leads described hereinabove.

What have been described above are examples and embodiments of the devices and methods described and disclosed herein. It is, of course, not possible to describe every conceivable combination of components or methodologies for purposes of describing the invention, but one of ordinary skill in the art will recognize that many further combinations and permutations of the devices and methods described and disclosed herein are possible. Accordingly, the devices and methods described and disclosed herein are intended to embrace all such alterations, modifications and variations that fall within the scope of the appended claims. In the claims, unless otherwise indicated, the article “a” is to refer to “one or more than one.”

The foregoing outlines features of several embodiments so that those skilled in the art may better understand the detailed description set forth herein. Those skilled in the art will now understand that many different permutations, combinations and variations of hearing aid 10 fall within the scope of the various embodiments. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

After having read and understood the present specification, those skilled in the art will now understand and appreciate that the various embodiments described herein provide solutions to long-standing problems, both in the use of electrophysiological mapping systems and in the use of cardiac ablation systems.

I claim:

1. A multiple configuration electrophysiological (EP) mapping catheter, comprising:
an elongated catheter body comprising a proximal portion, a distal portion, and a distal tip;
an electrode deployment and control mechanism located near or at the proximal portion of the catheter body;
a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof;
wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first initial deployment configuration suitable for pulmonary vein isolation (PV) EP mapping; (b) a second intermediate deployment fan or paddle configuration suitable for high-resolution EP mapping; and (c) a third fully or nearly fully deployed basket configuration suitable for medium-resolution EP mapping, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and (iii) in the third configuration the electrode mapping assembly is deployed by the user a third distance from the distal portion of the catheter body, and further wherein the first distance is less than the second distance, the second distance is less than the third distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening.

2. The multiple configuration EP mapping catheter of claim 1, wherein the catheter is further configured to permit portions of the catheter body located proximally from the distal tip to be moved by the user away from the longitudinal axis of the basket in the direction of and through the opening.

3. The multiple configuration EP mapping catheter of claim 1, wherein the catheter is further configured to permit portions of the catheter body located proximally from the distal tip to be moved by the user away from the longitudinal axis of the basket in the direction of and outside the opening.

4. The multiple configuration EP mapping catheter of claim 1, wherein the distal tip of the catheter is configured to be steerable or bent by the user.

5. The multiple configuration EP mapping catheter of claim 1, further comprising an outer slidable sheath configured to permit deployment of the electrode mapping assembly from the distal tip of the catheter.

6. The multiple configuration EP mapping catheter of claim 5, wherein the outer slidable sheath is steerable.

7. The multiple configuration EP mapping catheter of claim 6, wherein the steerable sheath comprises a steerable distal end.

8. The multiple configuration EP mapping catheter of claim 1, wherein the electrode mapping assembly comprises between 4 splines and 12 splines.

9. The multiple configuration EP mapping catheter of claim 1, wherein each spline has attached thereto, mounted thereon or formed therein between 1 and 16 electrodes.

10. The multiple configuration EP mapping catheter of claim 1, wherein the distal ends of adjoining splines forming pairs of splines are joined or connected to one another.

11. The multiple configuration EP mapping catheter of claim 1, further comprising one or more navigation elements, navigation coils, navigation markers or navigation electrodes.

12. The multiple configuration EP mapping catheter of claim 1, wherein the shape memory material comprises one or more of Nitinol, a shape memory metal, a shape memory alloy, a shape memory polymer, a shape memory composite, or a shape memory hybrid.

13. The multiple configuration EP mapping catheter of claim 1, wherein at least one spline in the electrode mapping assembly comprises laminated materials.

14. The multiple configuration EP mapping catheter of claim 1, wherein the mapping electrode assembly is deployed by pushing the mapping electrode assembly out of the distal end of the catheter using the electrode deployment and control mechanism.

15. The multiple configuration EP mapping catheter of claim 1, further comprising a tissue ablation mechanism located at or near the distal tip of the catheter.

16. The multiple configuration EP mapping catheter of claim 1, wherein a spatial resolution provided by the electrodes in the electrode mapping assembly and an associated spacing between splines changes in accordance with the first, second and third configurations thereof.

17. The multiple configuration EP mapping catheter of claim 1, wherein a diameter of the arms of the electrode mapping assembly ranges between about 6 mm and about 14 mm when the electrode mapping assembly is deployed in the first configuration.

18. The multiple configuration EP mapping catheter of claim 1, wherein a diameter of the arms of the electrode mapping assembly ranges between about 6 mm and about 14 mm when the electrode mapping assembly is deployed in the first configuration.

19. The multiple configuration EP mapping catheter of claim 1, wherein a diameter of the arms of the electrode mapping assembly ranges between about 10 mm and about 20 mm when the electrode mapping assembly is deployed in the first configuration.

20. The multiple configuration EP mapping catheter of claim 1, wherein a length of each tendon or chord ranges between about 6 mm and about 20 mm.

21. The multiple configuration EP mapping catheter of claim 1, wherein the electrodes are one or more of unipolar electrodes and bipolar electrodes.

22. The multiple configuration EP mapping catheter of claim 1, wherein spacing between adjoining electrodes located on the same spline ranges between about 0.5 mm and about 1 mm, between about 0.25 mm and about 2 mm, between about 6 mm and about 20 mm, between about 8 mm and about 18 mm, or between about 10 mm and about 15 mm.

23. The multiple configuration EP mapping catheter of claim 1, wherein the basket in the third configuration has an outer diameter ranging between about 20 mm and about 200 mm, between about 30 mm and about 100 mm in diameter, between about 40 mm and about 80 mm in diameter, or between about 50 mm and about 70 mm, or is about 50 mm, about 60 mm or about 70 mm.

24. A method of deploying a multiple configuration electrophysiological (EP) mapping catheter in a patient, the catheter comprising an elongated catheter body comprising a proximal portion, a distal portion, and a distal tip, an electrode deployment and control mechanism located near or at the proximal portion of the catheter body, a deployable multiple configuration electrode mapping assembly operably connected to the electrode deployment and control mechanism, the electrode mapping assembly comprising a plurality of electrodes and a plurality of pairs of splines, each spline having a proximal end and a distal end, the splines of each pair being connected at their distal ends by connecting members to form distal arms, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip, some but not all adjoining pairs of splines and the arms formed thereby being connected to one another by tendons or chords located at or near the distal ends thereof, wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into any two or more of the following configurations: (a) a first initial deployment configuration suitable for pulmonary vein isolation (PV) EP mapping; (b) a second intermediate deployment fan or paddle configuration suitable for high-resolution EP mapping; and (c) a third fully or nearly fully deployed basket configuration suitable for medium-resolution EP mapping, the basket configuration having an imaginary central longitudinal axis associated therewith when the basket is deployed in an unobstructed and unconfined space, and further wherein: (i) in the first configuration the electrode mapping assembly is deployed by the user a first distance from the distal portion of the catheter body; (ii) in the second configuration the electrode mapping assembly is deployed by the user a second distance from the distal portion of the catheter body; and (iii) in the third configuration the electrode mapping assembly is deployed by the user a third distance from the distal portion of the catheter body, and further wherein the first distance is less than the second distance, the second distance is less than the third distance, an opening is located between at least portions of two adjoining splines in the electrode mapping assembly, no chord or tendon is located within at least portions of the opening such that portions of the catheter body located proximally from the distal tip can be moved by a user away from the longitudinal axis of the basket in a direction of the opening, the method comprising two or more of:
(1) deploying the electrode mapping assembly into the first configuration inside or near the patient's heart;
(2) deploying the electrode mapping assembly into the second configuration inside or near the patient's heart, and
(3) deploying the electrode mapping assembly into the third configuration inside or near the patient's heart.

25. The method of claim 24, wherein the distal tip of the catheter is configured to be steerable or bent by the user, and the user bends or steers the distal tip of the catheter inside or near the patient's heart.

26. The method of claim 24, further comprising acquiring EP signals from the patient using electrodes in the deployed electrode mapping assembly.

27. The method of claim 26, further comprising processing the acquired EP signals so that the signals may be interpreted by the user.

28. The method of claim 27, further comprising redeploying the electrode mapping assembly into a different configuration or location within or near the patient's heart based upon results provided by the processed EP signals.

29. The method of claim 24, further comprising changing the configuration of the electrode mapping assembly from one of the first, second and third configurations to a different configuration.

30. The method of claim 24, further comprising deploying the mapping electrode assembly by pushing the mapping electrode assembly out of the distal end of the catheter using the electrode deployment and control mechanism.

31. The method of claim 27, further comprising ablating tissue at a location in or near the patient's heart, the location being identified using the processed EP signals.

32. An electrophysiological (EP) mapping catheter, comprising:
an elongated catheter body comprising a proximal portion, a distal portion, and a distal tip;
an electrode deployment and control mechanism located near or at the proximal portion of the catheter body;
a deployable multiple configuration electrode mapping assembly operably connected to the electrode deployment and control mechanism, the electrode mapping assembly comprising a plurality of electrodes and a plurality of splines, each spline having a proximal end and a distal end, the electrodes being mounted on or connected to at least some of the splines, at least some of the splines comprising a shape memory material, at least the distal end of each spline being configured to bend or be bent backwardly from the distal tip towards more proximal portions of the catheter body as the plurality of splines is deployed from or near the distal tip;
wherein at least major portions of the electrode mapping assembly are configured to fit within the distal portion of the catheter body when the electrode assembly is in an undeployed configuration, the electrode assembly further being configured to be controllably deployed and advanced from the distal tip of the catheter by a user operating the electrode deployment and control mechanism into at least one of the following configurations: (a) a first circular, semi-circular, oval, elliptical, or lasso-like configuration suitable for pulmonary vein isolation (PV) EP mapping; (b) a second fan-shaped configuration of the mapping electrode assembly suitable for acquiring high-resolution EP data; and (c) a third basket configuration suitable for acquiring medium-resolution EP data.
